(19) United States
(12) Patent Application Publication
(10) Pub. No.: US 2024/0043891 A1
(43) Pub. Date: Feb. 8, 2024

(54) A DFL-PRODUCING STRAIN

(71) Applicant: GLYCOM A/S, Hørsholm (DK)

(72) Inventor: Margit PEDERSEN, Roskilde (DK)

(21) Appl. No.: 18/258,770

(22) PCT Filed: Dec. 21, 2021
(86) PCT No.: PCT/EP2021/086932

§ 371 Date: Jun. 21, 2023

(30) Foreign Application Priority Data
Dec. 22, 2020 (DK) PA 2020 01450

(51) Int. Cl. C12P 19/18; C12N 9/10

(52) U.S. Cl. CPC C12P 19/18; C12N 9/1051; C12Y 204/01069; C12Y 204/01065

(57) ABSTRACT

The present invention relates to a genetically modified cell expressing an α-1,2-fucosyltransferase and an α-1,3-fucosyltransferase, and a transporter protein of the major facilitator superfamily (MFS) and to a method for recombinant production of human milk oligosaccharides (HMOs) using said genetically modified cell. More particularly, the invention provides a method for recombinant production of and a genetically modified cell capable of producing difucosyllactose (DFL) as the most abundant HMO, with a relatively low content of 3-fucosyllactose (3FL) and/or 2′-fucosyllactose (2′FL).

### FIELD OF THE INVENTION

The present invention relates to the field of recombinant production of biological molecules in host cells. In particular, it relates to a method for recombinant production of human milk oligosaccharides (HMOs) using genetically modified cells expressing an α-1,2-fucosyltransferase and an α-1,3-fucosyltransferase, and, optionally, a protein of the major facilitator superfamily (MFS). More particularly, the invention provides a method for recombinant production of and a genetically modified cell capable of producing mainly difucosyllactose (DFL) with a relatively low content of 3-fucosyllactose (3FL) and 2′-fucosyllactose (2′FL).

### BACKGROUND OF THE INVENTION

Human milk represents a complex mixture of carbohydrates, fats, proteins, vitamins, minerals and trace elements. The by far most predominant fraction is represented by carbohydrates, which can be further divided into lactose and more complex oligosaccharides (Human milk oligosaccharides, HMO). Whereas lactose is used as an energy source, the complex oligosaccharides are not metabolized by the infant. The fraction of complex oligosaccharides accounts for up to 1/10 of the total carbohydrate fraction and consists of probably more than 150 different oligosaccharides. The occurrence and concentration of these complex oligosaccharides are specific to humans and thus cannot be found in large quantities in the milk of other mammals, like for example domesticated dairy animals.

The most prominent oligosaccharides are 2′-fucosyllactose and 3-fucosyllactose which together can contribute up to ⅓ of the total HMO fraction. Further prominent HMOs present in human milk are lacto-N-tetraose, lacto-N-neotetraose and the lacto-N-fucopentaose I. Besides these neutral oligosaccharides, also acidic HMOs can be found in human milk, such as 3′-sialyllactose, 6′-sialyllactose and 3-fucosyl-3′-sialyllactose, disialyl-lacto-N-tetraose etc. These fucosyl- and sialyl-structures are closely related to epitopes of epithelial cell surface glycoconjugates, i.e., the Lewis histoblood group antigens, such as Lewis x (LeX) which are considered hallmarks of cancer pathogenesis. The structural homology of HMOs to epithelial epitopes accounts for their protective properties against bacterial pathogens.

The existence of complex oligosaccharides in human milk has been known for a long time and the physiological functions of these oligosaccharides have been subject to medicinal research for many decades. For some of the more abundant human milk oligosaccharides, specific functions have already been identified.

Besides local effects in the intestinal tract, HMOs have been shown to elicit systemic effects in infants by entering the systemic circulation. Also, the impact of HMOs on protein-carbohydrate interactions, e.g., selectin-leukocyte binding, can modulate immune responses and reduce inflammatory responses. In addition, it has become more and more recognized that HMOs represent a key substrate for the development of infants' microbiome.

Human milk oligosaccharides (HMOs) constitute the third largest solid component in human milk and are highly resistant to enzymatic hydrolysis. As a consequence, a substantial fraction of HMOs remains largely undigested and unabsorbed, which enables their passage through to the colon. In the colon, HMOs may serve as substrates to shape the gut ecosystem by selectively stimulating the growth of specific saccharolytic bacteria. This selectivity is viewed as beneficial for both infants and adults since strains of Bifidobacterium species are believed to have a positive effect on gut health (Chichlowski M. et al., (2012) J. Pediatr. Gastroenterol. Nutr. 5:251-258; Elison E. et al., (2016) Brit J. Nutr, 116: 1356-1368).

Besides their prebiotic properties, HMOs have been linked to additional positive effects, which expands their field of application (Kunz C. et al., (2014) Food Oligosaccharides: Production, Analysis and Bioactivity, 1st Edition, p 5-20, Eds. Moreno J. and Luz Sanz M., John Wiley & Sons, Ltd).

The obvious health benefits of HMOs have enabled their approval for use in foods, such as infant formulas and foods, and for consumer health products.

Due to the well-studied beneficial properties of prebiotic oligosaccharides, in particular of HMOs, connected with their limited availability, an effective commercial, i.e. large scale production is highly desirable.

Biotechnological production of HMOs is a valuable cost-efficient and large-scale way of HMO manufacturing. It relies on genetically engineered bacteria or yeast constructed so as to express the glycosyltransferases needed for synthesis of the desired oligosaccharides and takes advantage of the bacteria's innate pool of nucleotide sugars as HMO precursors.

Recent developments in biotechnological production of HMOs have made it possible to overcome certain inherent limitations of bacterial expression systems. For example, WO2012112777 describes HMO-producing bacterial cells may be genetically modified to produce fucosylated oligosaccharides by potentially having both alpha-1,2 fucosyltransferase and alpha-1,3 fucosyltransferase activity and to increase the limited intracellular pool of nucleotide sugars in the bacteria. The cell produces a mixture of 2′FL, 3FL and DFL, but only with low amount of DFL. WO2016040531 discloses a specific alpha-1,3 fucosyltransferase with improved activity in the fucosylated HMO production. WO2010142305 and WO2017042382 describe use of exporters to facilitate secretion of synthesized HMOs into the extracellular media. Further, expression of genes of interest in recombinant cells may be regulated by using particular promoters or other gene expression regulators, like e.g., as recently described in WO2019123324.

The approach described in WO2010142305 and WO2017042382 has the advantage that it allows to reduce the metabolic burden inflicted on the producing cell by high levels of recombinant gene expression, e.g., using methods of WO2012112777, WO2016040531 or WO2019123324. This approach attracts growing attention in recombinant HMO-producing cells engineering, e.g., recently several new sugar transporter genes have been described that can facilitate efflux of recombinantly produced 2′-fucosyllactose (2′FL) WO2018077892 discloses setA and YberC, US201900323053 and US201900323052 relate to yeast transporters.

However, at present, there is still a need for providing recombinant methods capable of effectively producing specific HMOs.

### SUMMARY OF THE INVENTION

The present invention relates to a genetically modified cell capable of producing HMOs. The HMO produced is primarily DFL. Preferably the DFL is produced in an amount corresponding to more than 50%, such as 60%, of the total HMOs produced. The other HMOs produced are primarily selected from 3FL and 2′FL and combinations thereof.

An aspect of the invention is a genetically modified cell comprising a heterologous, recombinant and/or synthetic nucleic acid encoding
a. an α-1,2-fucosyltransferase, and
b. an α-1,3-fucosyltransferase
c. a transporter protein selected from the major facilitator superfamily (MFS).

In one embodiment, the genetically modified cell according to the present invention comprises a heterologous, recombinant and/or synthetic nucleic acid encoding an α-1,2-fucosyltransferase which is a futC or wbgL gene and a nucleic acid encoding an α-1,3-fucosyltransferase which is selected from a futA gene or a fucT gene or moumou gene.

Typically, the genetically modified cell with the MFS transporter protein produces at least 5% w/w more DFL compared to the same cell without the MFS transporter protein. Typically, the genetically modified cell produces 50% w/w or more, such as 60% w/w, such as 65% w/w or more, or 70% w/w or more of the HMOs produced by the cell described herein is difucosyllactose (DFL) and at the most 35% w/w of the total amount of the HMOs produced in the cell is 3-fucosyllactose (3FL) and/or 2′-fucosyllactose (2′FL), such as at the most 30%, such as at the most 20% w/w, such as at the most 15% w/w, at the most 10% w/w, at the most 5% w/w, at the most 2.5% w/w or at the most 1% w/w of the total amount of the HMOs produced in the cell is 3-fucosyllactose. Further, at the most 30% w/w such as at the most 20% w/w, such as at the most 15% w/w, at the most 10% w/w, at the most 5% w/w, at the most 2.5% w/w, or at the most 1% w/w of the total amount of the HMOs produced in the cell is 2′-fucosyllactose (2′FL).

In a presently preferred embodiment, the genetically modified cell further comprises a heterologous, recombinant and/or synthetic nucleic acid encoding a transporter protein selected from the major facilitator superfamily (MFS). The transporter protein can consist of SEQ ID NO: 1 (marc), or a functional homologue thereof which amino acid sequence is at least 80% identical, preferably at least 85% identical, more preferably at least 90% identical to SEQ ID NO: 1, or SEQ ID NO: 2 (nec), or a functional homologue thereof which amino acid sequence is at least 80% identical, preferably at least 85% identical, more preferably at least 90% identical to SEQ ID NO: 2, or SEQ ID NO: 3 (vag), or a functional homologue thereof which amino acid sequence is at least 80% identical, preferably at least 85% identical, more preferably at least 90% identical to SEQ ID NO: 3, or SEQ ID NO: 42 (fred), or a functional homologue thereof which amino acid sequence is at least 80% identical, preferably at least 85% identical, more preferably at least 90% identical to SEQ ID NO: 42, or or SEQ ID NO: 43 (bad), or a functional homologue thereof which amino acid sequence is at least 80% identical, preferably at least 85% identical, more preferably at least 90% identical to SEQ ID NO: 43.

A genetically modified cell according to the present invention is a microbial cell, preferably Escherichia coli. Said cell can further comprise a heterologous, recombinant and/or synthetic regulatory element comprising a nucleic sequence for the regulation of the expression of the heterologous, recombinant and/or synthetic nucleic acid, such as a promoter nucleic sequence, such as a lac promoter, Plac, or a mglB promoter, PmglB, or a glp promoter, PglpF, or any variation thereof. Preferably, the promoter nucleic sequence is PglpF, PmglB, or a variant thereof.

The present invention further relates to a method for the production of one or more oligosaccharides, wherein the primary oligosaccharide produced is difucosyllactose (DFL), the method comprising the steps of:
(i) providing a genetically modified cell capable of producing an HMO, wherein said cell comprises a heterologous, recombinant and/or synthetic nucleic acid encoding
   a. an α-1,2-fucosyltransferase, and
   b. an α-1,3-fucosyltransferase, and
   c. a transporter protein selected from the major facilitator superfamily (MFS)
(ii) culturing the cell according to (i) in a suitable cell culture medium to produce said HMO; and
(iii) harvesting one or more HMOs produced in step (ii).

Typically, said heterologous, recombinant and/or synthetic nucleic acid encoding an α-1,2-fucosyltransferase is a futC and/or wbgL gene, or a functional homologue thereof, and said heterologous, recombinant and/or synthetic nucleic acid encoding an α-1,3-fucosyltransferase is selected from a futA gene and/or a fucT gene or moumou gene, or a functional homologue of a futA gene and/or a fucT gene or moumou gene.

Said genetically modified cell can further comprise a heterologous, recombinant and/or synthetic nucleic acid encoding a transporter protein selected from the major facilitator superfamily (MFS); such as, but not limited to, marc, nec, vag, fred or bad.

At the most 35% w/w of the total amount of the HMOs produced by a method described herein is 3-fucosyllactose (3FL), such as at the most 20% w/w, at the most 15% w/w, at the most 10% w/w, at the most 5% w/w, at the most 2.5% w/w, or at the most 1% w/w. In one aspect, the method described herein does essentially not produce 3-fucosyllactose (3FL) or does only produce very little 3-fucosyllactose (3FL), such as essentially 0.1-0% w/w of the total amount of the HMOs produced.

Further, at the most 35% w/w of the total amount of the HMOs produced by a method described herein is 2′-fucosyllactose (2′FL), such as at the most 20% w/w, at the most 15% w/w, at the most 10% w/w, at the most 5% w/w, at the most 2.5% w/w, or at the most 1% w/w.

In one aspect, the culturing of the cell in step (ii) is conducted at low lactose conditions, such as at conditions having <5 g lactose/l culture medium.

The invention further relates to the use of a genetically modified cell according to the present invention for the production of one or more HMO, wherein at least 50% w/w, such as at least 60% w/w, such as at least 65% w/w, such as at least 70% w/w of the HMOs produced in the cell is difucosyllactose (DFL).

Other aspects and advantageous features of the present invention are described in detail and illustrated by non-limiting working examples below.

### DEFINITIONS AND ABREVIATIONS

In cell biology and protein biochemistry, heterologous expression means that a protein is experimentally put into a cell that does not normally make that protein.

Recombinant DNA molecules are DNA molecules formed by laboratory methods of genetic recombination that bring together genetic material from multiple sources, creating sequences that would not otherwise be found in the genome.

A synthetic nucleic acid, such as but not limited to a synthetic promoter is a stretch of DNA designed and chemically synthesized. The synthetic promoter typically comprises a core-promoter region and multiple repeats or combinations of heterologous upstream regulatory elements (cis-motifs and/or TF-binding sites).

The terms “recombinant cell”, “recombinant cell line” or “recombinant strain” are in the present context used to mean a cell, cell line or strain into which recombinant DNA has been introduced, either stably or transiently, i.e. in which genetic recombination has taken place.

In the present context, a genetically modified cell is a cell that has been genetically altered to express heterologous, recombinant and/or synthetic DNA. In the present context, the terms “genetically modified” and “genetically engineered” are used interchangeably. A recombinant cell, cell line or strain is a genetically modified cell, cell line or strain.

The term “genetically engineered” and/or “genetically modified” as used herein refers to the modification of the microbial cell's genetic make-up using molecular biological methods. The modification of the microbial cell's genetic make-up may include the transfer of genes within and/or across species boundaries, inserting, deleting, replacing and/or modifying nucleotides, triplets, genes, open reading frames, promoters, enhancers, terminators and other nucleotide sequences mediating and/or controlling gene expression. The modification of the microbial cell's genetic make-up aims to generate a genetically modified organism possessing particular, desired properties.

Genetically engineered microbial cells can contain one or more genes that are not present in the native (not genetically engineered) form of the cell. Techniques for introducing exogenous nucleic acid molecules and/or inserting exogenous nucleic acid molecules (recombinant, heterologous) into a cell's hereditary information for inserting, deleting or altering the nucleotide sequence of a cell's genetic information are known to the skilled artisan.

Genetically engineered microbial cells can contain one or more genes that are present in the native form of the cell, wherein said genes are modified and re-introduced into the microbial cell by artificial means. The term “genetically engineered” also encompasses microbial cells that contain a nucleic acid molecule being endogenous to the cell, and that has been modified without removing the nucleic acid molecule from the cell. Such modifications include those obtained by gene replacement, site-specific mutations, and related techniques.

The terms “recombinant nucleic acid sequence”, “recombinant gene/nucleic acid/DNA” are used interchangeably and refers to an artificial nucleic acid sequence (i.e. produced in vitro using standard laboratory methods for making nucleic acid sequences). recombinant nucleic acids may also be non-encoding promotor or other regulatory elements.

By the term “recombinant gene/nucleic acid/nucleotide sequence/DNA encoding” or “coding nucleic acid sequence” is meant an artificial nucleic acid sequence (i.e., produced in vitro using standard laboratory methods for making nucleic acid sequences) that comprises a set of consecutive, non-overlapping triplets (codons) which is transcribed into mRNA and translated into a polypeptide when placed under the control of the appropriate control sequences, i.e. promoter. The boundaries of the coding sequence are generally determined by a ribosome binding site located just upstream of the open reading frame at the 5′end of the mRNA, a transcriptional start codon (AUG, GUG or UUG), and a translational stop codon (UAA, UGA or UAG). A coding sequence can include, but is not limited to, genomic DNA, cDNA, synthetic, and heterologous, recombinant and/or synthetic sequences. The term “nucleic acid” includes RNA, DNA and cDNA molecules. It is understood that, as a result of the degeneracy of the genetic code, a multitude of nucleotide sequences encoding a given protein may be produced. The term nucleic acid is used interchangeably with the term “polynucleotide”. An “oligonucleotide” is a short chain nucleic acid molecule.

The term “nucleotide sequence encoding” generally refers to any polyribonucleotide or polydeoxyribonucleotide, which may be unmodified RNA or DNA or modified RNA or DNA, and generally represents the portion of a gene which encodes a certain polypeptide or protein. The term includes, without limitation, single- and double-stranded DNA, DNA that is a mixture of single- and double-stranded regions or single-, double- and triple-stranded regions, single- and double-stranded RNA, and RNA that is mixture of single- and double-stranded regions, hybrid molecules comprising DNA and RNA that may be single-stranded or, more typically, double-stranded, or triple-stranded regions, or a mixture of single- and double-stranded regions. The term also encompasses polynucleotides that include a single continuous region or discontinuous regions encoding the polypeptide (for example, interrupted by integrated phage or an insertion sequence or editing) together with additional regions that also may contain coding and/or non-coding sequences.

The term “heterologous” as used herein refers to a polypeptide, amino acid sequence, nucleic acid molecule or nucleotide sequence that is foreign to a cell or organism, i.e. to a polypeptide, amino acid sequence, nucleic acid molecule or nucleotide sequence that does not naturally occurs in said cell or organism. A “heterologous sequence” or a “heterologous nucleic acid” or “heterologous polypeptide”, as used herein, is one that originates from a source foreign to the particular host cell (e.g. from a different species), or, if from the same source, is modified from its original form. Thus, a heterologous nucleic acid operably linked to a promoter is from a source different from that from which the promoter was derived, or, if from the same source, is modified from its original form. The heterologous sequence may be stably introduced, e.g. by transfection, transformation, conjugation or transduction, into the genome of the host microbial host cell, thus representing a genetically modified host cell. Techniques may be applied which will depend on the host cell the sequence is to be introduced. Various techniques are known to a person skilled in the art and are, e.g., disclosed in Sambrook et al., Molecular Cloning: A Laboratory Manual, 2nd Ed., Cold Spring Harbor Laboratory Press, Cold Spring Harbor, N.Y. (1989).

As used herein, the terms “nucleic acid” and “polynucleotide” refer to a deoxyribonucleotide or ribonucleotide polymer in either single- or double-stranded form, and unless otherwise limited, encompasses known analogues of natural nucleotides that hybridize to nucleic acids in a manner similar to naturally occurring nucleotides. Unless otherwise indicated, a particular nucleic acid sequence includes the complementary sequence thereof.

The term “functional gene” as used herein, refers to a nucleic acid molecule comprising a nucleotide sequence which encodes a protein or polypeptide, and which also contains regulatory sequences operably linked to said protein-coding nucleotide sequence such that the nucleotide sequence which encodes the protein or polypeptide can be expressed in/by the microbial cell bearing said functional gene. Thus, when cultivated at conditions that are permissive for the expression of the functional gene, said functional gene is expressed, and the microbial cell expressing said functional gene typically comprises the protein or polypeptide that is encoded by the protein coding region of the functional gene.

The term “operably linked” refers to a functional relationship between two or more nucleic acid (e.g., DNA) segments. Typically, it refers to the functional relationship of a transcriptional regulatory sequence to a transcribed sequence. Operably linked means that there is a functional linkage between a nucleic acid expression control sequence (such as a promoter, signal sequence, or array of transcription factor binding sites) and a second nucleic acid sequence, wherein the expression control sequence affects transcription and/or translation of the nucleic acid corresponding to the second sequence. For example, a promoter sequence is operably linked to a coding sequence if it stimulates or modulates the transcription of the coding sequence in an appropriate host cell or other expression system. Generally, promoter transcriptional regulatory sequences that are operably linked to a transcribed sequence are physically contiguous to the transcribed sequence, i.e., they are cis-acting.

The term “overexpression” or “overexpressed” as used herein refers to a level of enzyme or polypeptide expression that is greater than what is measured in a wild-type cell of the same species as the host cell that has not been genetically altered.

In the context of the invention, the term “oligosaccharide” means a saccharide polymer containing a number of monosaccharide units. The term “oligosaccharide” as used herein refers to a saccharide molecule consisting of three to twenty monosaccharide residues, wherein each of said monosaccharide residues is bound to at least one other of said monosaccharide units by a glycosidic linkage. The oligosaccharide may be a linear chain of monosaccharide residues or a branched chain of monosaccharide residues.

In some embodiments, preferred oligosaccharides are saccharide polymers consisting of three or four monosaccharide units, i.e., trisaccharides or tetrasaccharides. Preferable oligosaccharides of the invention are human milk oligosaccharides (HMOs).

The term “human milk oligosaccharide” or “HMO” in the present context means a complex carbohydrate found in human breast milk (for reference, see Urashima et al.: Milk Oligosaccharides. Nova Science Publisher (2011); or Chen, Adv. Carbohydr. Chem. Biochem. 72, 113 (2015)). The HMOs have a core structure comprising a lactose unit at the reducing end that can be elongated by one or more beta-N-acetyl-lactosaminyl and/or one or more beta-lacto-N-biosyl units, and this core structure can be substituted by an alpha-L-fucopyranosyl and/or an alpha-N-acetyl-neuraminyl (sialyl) moiety.

To date, the structures of at least 115 HMOs have been determined (see Urashima et al.: Milk Oligosaccharides, Nova Biomedical Books, New York, 2011, ISBN: 978-1-61122-831-1), and considerably more are probably present in human milk.

Non-acidic (or neutral) HMOs are devoid of a sialyl residue, and the acidic HMOs have at least one sialyl residue in their structure. The non-acidic (or neutral) HMOs can be fucosylated or non-fucosylated.

Examples of such neutral non-fucosylated HMOs include lacto-N-triose 2 (LNT-2) lacto-N-tetraose (LNT), lacto-N-neotetraose (LNnT), lacto-N-neohexaose (LNnH), para-lacto-N-neohexaose (pLNnH), para-lacto-N-hexaose (pLNH) and lacto-N-hexaose (LNH).

Examples of neutral fucosylated HMOs include 2′-fucosyllactose (2′FL), lacto-N-fucopentaose I (LNFP-I), lacto-N-difucohexaosel (LNDFH-I), 3-fucosyllactose (3FL), difucosyllactose (DFL), lacto-N-fucopentaose II(LNFP-II), lacto-N-fucopentaose III (LNFP-III), lacto-N-difucohexaose III (LNDFH-III), fucosyl-lacto-N-hexaose II(FLNH-II), lacto-N-fucopentaose V (LNFP-V), lacto-N-difucohexaose II (LNDFH-II), fucosyl-lacto-N-hexaose I (FLNH-I), fucosyl-para-lacto-N-hexaose I (FpLNH-I), fucosyl-para-lacto-N-neohexaose II (F-pLNnH II) and fucosyl-lacto-N-neohexaose (FLNnH).

Examples of acidic HMOs include 3′-sialyllactose (3′-SL), 6′-sialyllactose (6′-SL), 3-fucosyl-3′-sialyllactose (FSL), 3′-O-sialyllacto-N-tetraose a (LST a), fucosyl-LST a (FLST a), 6′-O-sialyllacto-N-tetraose b (LST b), fucosyl-LST b (FLST b), 6′-O-sialyllacto-N-neotetraose (LST c), fucosyl-LST c (FLST c), 3′-O-sialyllacto-N-neotetraose (LST d), fucosyl-LST d (FLST d), sialyl-lacto-N-hexaose (SLNH), sialyl-lacto-N-neohexaose I (SLNH-I), sialyl-lacto-N-neohexaose II (SLNH-II) and disialyl-lacto-N-tetraose (DSLNT).

In the context of the present invention lactose is not regarded as an HMO species.

The term “cultivating” (or “culturing” or “cultivation”, also termed “fermentation”) in the present context means growing a bacterial cell in a medium and under conditions permissive and suitable for the production of the desired oligosaccharide(s). Propagation of bacterial expression cells in a controlled bioreactor are methods known in the industry A couple of suitable bacterial host cells as well as mediums and conditions for their cultivation will be readily available for one skilled in the art upon reading the disclosure of this invention in connection with the skilled person's technical and expert background.

As used herein, the term “recovering” means isolating, harvesting, purifying, collecting or otherwise separating from the host microorganism culture the oligosaccharide(s) produced by the host microorganism.

The term “enzymatic activity” as used herein is meant to comprise any molecule displaying enzymatic activity, in particular a protein, and acting as a catalyst to bring about a specific biochemical reaction while remaining unchanged by the reaction. In particular, proteins with enzymatic activities are meant to be comprised by this term, which are able to convert a substrate into a product.

In enzymatic reactions, the molecules at the beginning of the process, called substrates, are converted into different molecules, called products. Almost all chemical reactions in a biological cell need enzymes in order to occur at rates sufficient for life. Since enzymes are selective for their substrates and speed up only a few reactions from among many possibilities, the set of enzymes made in a cell determines which metabolic pathways occur in that cell.

The term “variant(s)” as used herein, refers to a polynucleotide or polypeptide that differs from a reference polynucleotide or polypeptide respectively, but retains the essential (enzymatic) properties of the reference polynucleotide or polypeptide, also termed functional variant(s). A typical variant of a polynucleotide differs in nucleotide sequence from another, reference polynucleotide. Changes in the nucleotide sequence of the variant may or may not alter the amino acid sequence of a polypeptide encoded by the reference polynucleotide. Nucleotide changes may result in amino acid substitutions, additions, deletions, fusions and truncations in the polypeptide encoded by the reference sequence, as discussed below.

A variant and reference polypeptide may differ in amino acid sequence by one or more substitutions, additions, deletions in any combination. Generally, differences are limited so that the sequences of the reference polypeptide and the variant are closely similar overall and, in many regions, identical. A substituted or inserted amino acid residue may or may not be one encoded by the genetic code. A variant of a polynucleotide or polypeptide may be a naturally occurring such as an allelic variant, or it may be a variant that is not known to occur naturally. Non-naturally occurring variants of polynucleotides and polypeptides may be made by mutagenesis techniques, by direct synthesis, and by other recombinant methods known to the persons skilled in the art.

Accordingly, a “functional variant” of any of the genes/proteins disclosed therein, is meant to designate sequence variants of the genes/proteins still retaining the same or somewhat lesser activity of the gene or protein the respective fragment is derived from.

Within the scope of the present invention, also nucleic acid/polynucleotide and polypeptide polymorphic variants, alleles, mutants, and interspecies homologs are comprised by those terms, that have an nucleic acid/amino acid sequence that has greater than about 60% nucleic acid/amino acid sequence identity, 65%, 70%, 75%, 80%, 85%, 90%, preferably 91%, 92%, 93%, 94%, 95%, 96%, 97%, 98% or 99% or greater nucleic acid/amino acid sequence identity, preferably over a region of at least about 25, 50, 100, 200, 500, 1000, or more nucleic acid/amino acids, to a wildtype nucleic acid/amino acid sequence.

The term “sequence identity of [a certain] %” in the context of two or more nucleic acid or amino acid sequences means that the two or more sequences have nucleotides or amino acid residues in common in the given percent when compared and aligned for maximum correspondence over a comparison window or designated sequences of nucleic acids or amino acids (i.e. the sequences have at least 90 percent (%) identity). Percent identity of nucleic acid or amino acid sequences can be measured using a BLAST 2.0 sequence comparison algorithm with default parameters, or by manual alignment and visual inspection (see e.g. http://www.ncbi.nlm.nih.gov/BLAST/). This definition also applies to the complement of a test sequence and to sequences that have deletions and/or additions, as well as those that have substitutions. An example of an algorithm that is suitable for determining percent identity, sequence similarity and for alignment is the BLAST 2.2.20+ algorithm, which is described in Altschul et al. Nucl. Acids Res. 25, 3389 (1997). BLAST 2.2.20+ is used to determine percent sequence identity for the nucleic acids and proteins of the invention. Software for performing BLAST analyses is publicly available through the National Center for Biotechnology Information (http://www.ncbi.nlm.nih.gov/). Examples of commonly used sequence alignment algorithms are
CLUSTAL Omega (http://www.ebi.ac.uk/Tools/msa/clustalo/),
EMBOSS Needle (http://www.ebi.ac.uk/Tools/psa/emboss_needle/),
MAFFT (http://mafft.cbrc.jp/alignment/server/), or
MUSCLE (http://www.ebi.ac.uk/Tools/msa/muscle/).

Preferably, for purposes of the present invention, the sequence identity between two amino acid sequences is determined using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends Genet. 16: 276-277), preferably version 5.0.0 or later (available at https://www.ebi.ac.uk/Tools/psa/emboss needle/). The parameters used are gap open penalty of 10, gap extension penalty of 0.5, and the EBLOSUM62 (EMBOSS version of 30 BLOSUM62) substitution matrix. The output of Needle labeled “longest identity” (obtained using the—nobrief option) is used as the percent identity and is calculated as follows: (Identical Residues×100)/(Length of Alignment−Total Number of Gaps in Alignment)

Preferably, for purposes of the present invention, the sequence identity between two nucleotide sequences is determined using the Needleman-Wunsch algorithm (Needleman and Wunsch, 1 970, supra) as implemented in the Needle program of the EMBOSS package (EMBOSS: The European Molecular Biology Open Software Suite, Rice et al., 2000, Trends Genet. 16: 276-277), 10 preferably version 5.0.0 or later. The parameters used are gap open penalty of 10, gap extension penalty of 0.5, and the DNAFULL (EMBOSS version of NCBI NUC4.4) substitution matrix. The output of Needle labeled “longest identity” (obtained using the—nobrief option) is used as the percent identity and is calculated as follows: (Identical Deoxyribonucleotides×100)/(Length of Alignment−Total Number of Gaps in Alignment).

Unless otherwise specified, all technical and scientific terms used herein have the same meaning as commonly understood by one of ordinary skill in the art to which this invention belongs. Singleton et al. (1994) Dictionary of Microbiology and Molecular Biology, second edition, John Wiley and Sons (New York) provides one of skill with a general dictionary of many of the terms used in this invention. Although any methods and materials similar or equivalent to those described herein can be used in the practice or testing of the present invention, the preferred methods and materials are described. Most of the nomenclature and general laboratory procedures required in this application can be found in Sambrook et al., Molecular Cloning: A Laboratory Manual, Vol. 1-3, Cold Spring Harbor Laboratory, Cold Spring Harbor, New York (2012); Wilson K. and Walker J., Principles and Techniques of Biochemistry and Molecular Biology (2010), Cambridge University Press; or in Maniatise et al., Molecular Cloning A laboratory Manual, Cold Spring Harbor Laboratory (2012); or in Ausubel et al., Current protocols in molecular biology, John Wiley and Sohns (2010). The manuals are hereinafter referred to as “Sambrook et al.”, “Wilson & Walker”, “Maniatise et al.”, “Ausubel et al.”, correspondingly.

It is to be noticed that the term “comprising”, used in the claims, should not be interpreted as being restricted to the means listed thereafter; it does not exclude other elements or steps. It is thus to be interpreted as specifying the presence of the stated features, integers, steps or components as referred to, but does not preclude the presence or addition of one or more other features, integers, steps or components, or groups thereof. Thus, the scope of the expression “a device comprising means A and B” should not be limited to devices consisting only of components A and B. It means that with respect to the present invention, the only relevant components of the device are A and B. The term “comprising of” also includes the term “consisting of”.

Reference throughout this specification to “one embodiment” or “an embodiment” means that a particular feature, structure or characteristic described in connection with the embodiment is included in at least one embodiment of the present invention. Thus, appearances of the phrases “in one embodiment” or “in an embodiment” in various places throughout this specification are not necessarily all referring to the same embodiment but may. Furthermore, the particular features, structures or characteristics may be combined in any suitable manner, as would be apparent to one of ordinary skill in the art from this disclosure, in one or more embodiments.

In the description and drawings provided herein, numerous specific details are set forth. However, it is understood that embodiments of the invention may be practiced without these specific details. In other instances, well-known methods, structures and techniques have not been shown in detail in order not to obscure an understanding of this description.

Similarly, it should be appreciated that in the description of exemplary embodiments of the invention, various features of the invention are sometimes grouped together in a single embodiment, figure, or description thereof for the purpose of streamlining the disclosure and aiding in the understanding of one or more of the various inventive aspects. This method of disclosure, however, is not to be interpreted as reflecting an intention that the claimed invention requires more features than are expressly recited in each claim. Rather, as the following claims reflect, inventive aspects lie in less than all features of a single foregoing disclosed embodiment.

Thus, the claims following the detailed description are hereby expressly incorporated into this detailed description, with each claim standing on its own as a separate embodiment of this invention.

### DETAILED DESCRIPTION OF THE INVENTION

The present invention relates to a genetically modified cell for efficient production of specific HMOs and use of said genetically modified cell in a method of producing the HMOs. The HMOs produced are primarily DFL, which are produced in an amount corresponding to more than 50% w/w of the total HMOs produced, such as to at least 70% w/w of the total HMOs. The other HMOs produced are primarily selected from 3FL and 2′FL and combinations thereof.

A genetically modified cell capable of producing difucosyllactose (DFL) with a relatively low content of 3-fucosyllactose (3FL) and/or 2′-fucosyllactose (2′FL).

In particular, the present invention relates to a genetically modified cell enabled to synthesise an oligosaccharide, preferably a heterologous oligosaccharide, in particular a human milk oligosaccharide (HMO). Accordingly, a cell of the invention is modified to express a set of heterologous, recombinant and/or synthetic nucleic acids that are necessary for synthesis of one or more HMOs by the cells and which enable the cell to synthesise one or more HMOs, such as genes encoding one or more enzymes with glycosyltransferase activity as described below.

The oligosaccharide producing genetically modified cell of the invention can further be modified to comprise a heterologous, recombinant and/or synthetic nucleic acid sequence, preferably, a DNA sequence, encoding a putative MFS (major facilitator superfamily) transporter protein.

In general, the production of 2′FL requires that a genetically modified cell expresses an active α-1,2-fucosyltransferase enzyme; for the production of 3FL a genetically modified cell needs expression of an active α-1,3-fucosyltransferase enzyme. However, as seen from the examples herein, the primary HMO produced by the genetically modified cell of the present invention, which expresses both an active α-1,2-fucosyltransferase enzyme and an active α-1,3-fucosyltransferase enzyme, is mainly DFL.

As shown in the experimental section, it was found that the use of HMO producing recombinant cells that express an α-1,2-fucosyltransferase, an α-1,3-fucosyltransferase and, optionally, a transporter protein selected from the major facilitator superfamily (MFS), results in very distinct improvements of the HMO manufacturing process related both to fermentation and purification of the HMOs. The disclosed herein genetically modified cells and methods for HMO production provide both higher yields of total produced HMOs, lower by-product formation or by-product-to-product ratio, lower biomass formation per fermentation and facilitate simplified recovery of the HMOs during downstream processing of the fermentation broth.

In particular, surprisingly, the combined expression of a DNA sequence encoding an α-1,2-fucosyltransferase and an α-1,3-fucosyltransferase is herein demonstrated to result primarily in the production of difucosyllactose (DFL) with a relatively low content of 3-fucosyllactose (3FL) and/or 2′-fucosyllactose (2′FL). In particular an α-1,2-fucosyltransferase selected from FutC or WgbL or a functional variant thereof in combination with an α-1,3-fucosyltransferase selected from FutA or FucT or a functional variant thereof results in the production of DFL constituting at least 50% w/w of the total HMOs produced by the genetically modified cell.

Depending on the fermentation condition and the expression level of the enzymes, the combined expression of a DNA sequence encoding an α-1,2-fucosyltransferase and an α-1,3-fucosyltransferase results mainly in the production of difucosyllactose (DFL) with a relatively low content of 2′-fucosyllactose (2′FL) and less than 1% w/w of the total HMOs 3-fucosyllactose (3FL).

Thus, in one embodiment of the present invention, the combined expression of a DNA sequence encoding an α-1,2-fucosyltransferase and an α-1,3-fucosyltransferase results in the production of DFL (at least 70% w/w of the total HMOs), 2′FL (no more than 30% w/w of the total HMOs), and surprisingly essentially no 3FL.

Depending on the fermentation condition and the expression level of the enzymes, the combined expression of a DNA sequence encoding an α-1,2-fucosyltransferase and an α-1,3-fucosyltransferase results mainly in the production of difucosyllactose (DFL) with a relatively low content of 3-fucosyllactose (3FL) and less than 1% w/w of the total HMOs of 2′-fucosyllactose (2′FL).

Thus, in one embodiment of the present invention, the combined expression of a DNA sequence encoding an α-1,2-fucosyltransferase and an α-1,3-fucosyltransferase results in the production of DFL (at least 70% w/w of the total HMOs), 3FL (no more than 30% w/w of the total HMOs), and surprisingly essentially no 2′FL.

In one embodiment the α-1,2-fucosyltransferase is FutC or a functional variant with at least 90% identity such as at least 95% identity to SEQ ID NO: 37 in combination with the α-1,3-fucosyltransferase FutA or a functional variant with at least 90% identity such as at least 95% identity to SEQ ID NO: 38 or SEQ ID NO: 39. It is advantageously if the number of recombinant nucleic acid sequences encoding the FutC and FutA fucosyltransfeases in the cell are in the range of 1:1, such as 1:2, such as 1:3. In particular it is advantageous if the ratio of the active fucosyltransfeases, α-1,2-fucosyltransferase: α-1,3-fucosyltransferase, such as FutC:FutA or FutC:FucT or FutC:moumou ratio, in the cell are in the range of 1:1, such as 1:2, such as 1:3, such as 1:4, such as 1:5, such as 2:3 such as 2:5.

In addition, as disclosed in the experimental section, including the expression of a transporter protein selected from the major facilitator superfamily (MFS) enhances the selective production of DFL even further, such as with up to 25%, such as with 5%, 10% 15%, 20% or 25% w/w of the total HMOs.

In aspect of the present invention the introduction of the recombinant or heterologous MFS transporter protein increases the amount of DFL produced by the genetically modified cell compared to a cell that otherwise is identical except for the MFS transporter protein (control cell/strain). In one embodiment the genetically modified cell comprising an α-1,2-fucosyltransferase and an α-1,3-fucosyltransferase and a recombinant MFS transporter protein produces at least 5% more DFL compared to the same genetically modified cell without the MFS transporter protein. Preferably the introduction of a recombinant or heterologous MFS transporter protein increases the DFL production by at least 8%, 10% 15%, 20% or 25% compared to the control cell without the MFS transporter protein.

In one embodiment the combined expression of a DNA sequence encoding an α-1,2-fucosyltransferase and an α-1,3-fucosyltransferase and a transporter protein selected from the major facilitator superfamily (MFS) results in the production of DFL (at least 55% w/w, such as at least 65% w/w of the total HMOs) and 2′FL, 3FL (no more than 45% w/w of the total HMOs).

In one embodiment the combined expression of a DNA sequence encoding an α-1,2-fucosyltransferase and an α-1,3-fucosyltransferase and a transporter protein selected from the major facilitator superfamily (MFS) results in the production of DFL (at least 70% w/w of the total HMOs) and 2′FL, 3FL (no more than 30% w/w of the total HMOs).

In a particularly preferred embodiment, the combined expression of a DNA sequence encoding an α-1,2-fucosyltransferase and an α-1,3-fucosyltransferase and a transporter protein selected from the major facilitator superfamily (MFS) results in the production of DFL (at least 90% w/w of the total HMOs) and 2′FL, 3FL (no more than 10% w/w of the total HMOs).

In one aspect the present invention relates to a genetically modified cell capable of producing one or more Human Milk Oligosaccharides (HMOs), wherein said cell comprises a heterologous, recombinant and/or synthetic nucleic acid encoding
a. an α-1,2-fucosyltransferase, and
b. an α-1,3-fucosyltransferase.

Preferably, the primary HMOs produced by the cell is difucosyllactose (DFL), more preferably more than 50% w/w, such as more than 65% w/w, of the total HMO produced is difucosyllactose (DFL).

The term “primary HMO” is to be understood as the most abundant HMO in a mixture of HMOs produced by the genetically modified cell. So, in terms of DFL it means that there is more DFL than the individual amounts of e.g. 2′FL and 3FL, e.g. 40% DFL and 30% 2′FL and 30% 3FL will make DFL the primary HMO produced by the genetically modified cell.

In a preferred aspect, the present invention relates to a genetically modified cell capable of producing one or more Human Milk Oligosaccharides (HMOs), wherein said cell comprises a heterologous, recombinant and/or synthetic nucleic acid encoding
a. an α-1,2-fucosyltransferase,
b. an α-1,3-fucosyltransferase, and
c. a transporter protein selected from the major facilitator superfamily (MFS).

Preferably, the genetically modified cell with the MFS transporter protein produces at least 5% w/w more difucosyllactose (DFL) than the genetically modified without the MFS transporter protein.

In one aspect of the invention, the genetically modified cell produces at least 45%, such as at least 50% w/w DFL of the total HMOs, such as between 45-99%, such as at least 55, 60, 65, 70, 75, 80, 85, 90, 95 or 99% DFL. Preferably 70, 75, 80, 85, 90, 95 or 99% DFL.

In one aspect of the invention, the genetically modified cell produces at the most 45% w/w of 2′FL and/or 3FL the total HMOs, such as between 5-10%, 5-15%, 5-30%, 5-40%, such as at the most 0.5, 1, 5, 10, 5, 20, 25, 30, 35 or 40% 2′FL and/or 3FL.

In one aspect of the invention, the genetically modified cell produces at the most 5% w/w of the total HMOs, such as between 0-5%, such as at the most 0.1, 0.2, 0.3, 0.4, 0.5, 0.6, 0.7, 0.8, 0.9, 1, 1.5, 2, 2.5, 3, 3.5, 4, 4.5 or 5% 2′FL and/or 3FL.

A genetically modified cell of the present invention is typically a microbial cell, preferably a prokaryotic cell. Appropriate microbial cells include yeast cells, bacterial cells, archaebacterial cells, algae cells, and fungal cells.

The genetically modified microbial cell may be a bacterial cell, preferably a bacterial cell selected from the group consisting of Bacillus, Lactobacillus, Lactococcus, Enterococcus, Bitidobacterium, Sporoiactobacillus spp., Micromomospora spp., Micrococcus spp., Rhodococcus spp., and Pseudomonas. Suitable bacterial species are Bacillus subtilis, Bacillus licheniformis, Bacillus coagulans, Bacillus thermophilus, Bacillus laterosporus, Bacillus megaterium, Bacillus mycoides, Bacillus pumilus, Bacillus lentus, Bacillus cereus, Bacillus circulans, Bifidobacterium longum, Bifidobacterium infantis, Bifidobacterium bifidum, Citrobacter freundii, Clostridium cellulolyticum, Clostridium ljungdahlii, Clostridium autoethanogenum, Clostridium acetobutylicum, Corynebacterium glutamicum, Enterococcus faecium, Enterococcus thermophiles, Escherichia coli, Erwinia herbicola (Pantoea agglomerans), Lactobacillus acidophilus, Lactobacillus salivarius, Lactobacillus plantarum, Lactobacillus helveticus, Lactobacillus delbrueckii, Lactobacillus rhamnosus, Lactobacillus bulgaricus, Lactobacillus crispatus, Lactobacillus gasseri, Lactobacillus casei, Lactobacillus reuteri, Lactobacillusj ensenii, Lactococcus lactis, Pantoea citrea, Pectobacterium carotovorum, Proprionibacterium freudenreichii, Pseudomonas fluorescens, Pseudomonas aeruginosa, Streptococcus thermophiles and Xanthomonas campestris. A person skilled in the art will be aware of further bacterial strains when reading the present disclosure.

As exemplified in the experimental section, a presently preferred genetically modified microbial cell is an Escherichia coli cell.

The genetically engineered cell may be a yeast cell, preferably selected from the group consisting of Saccharomyces sp., in particular Saccharomyces cerevisiae, Saccharomycopsis sp., Pichia sp., in particular Pichia pastoris, Hansenula sp., Kluyveromyces sp., Yarrowia sp., Rhodotoruta sp., and Schizosaccharomyces sp.

The genetically engineered cell may be filamentous fungi such as Aspargillus sp, Fusarium sp or Thricoderma sp, exemplary species are A. niger, A. nidulans, A. oryzae, F. solani, F. graminearum and T. reesei.

A genetically modified cell may further comprise control sequences enabling the controlled overexpression of endogenous or heterologous, recombinant and/or synthetic sequences. As defined above, the term “control sequence” which herein is synonymously used with the expression “nucleic acid expression control sequence”, comprises promoter sequences, signal sequence, or array of transcription factor binding sites, which sequences affect transcription and/or translation of a nucleic acid sequence operably linked to the control sequences.

A nucleic acid sequence may be placed under the control of an inducible promoter, which is a promoter that directs expression of a gene where the level of expression is alterable by environmental or developmental factors such as, for example, temperature, pH, anaerobic or aerobic conditions, light, transcription factors and chemicals. Such promoters are referred to herein as “inducible” promoters, which allow one to control the timing of expression of the proteins used in the present invention. For E. coli and other bacterial host cells, inducible promoters are known to those of skill in the art.

The nucleic acid sequences as used in the present invention, may, e.g., be comprised in a vector which is to be stably transformed/transfected or otherwise introduced into host microorganism cells.

A great variety of expression systems can be used to produce polypeptides. Such vectors include, among others, chromosomal, episomal and virus-derived vectors, e.g., vectors derived from bacterial plasmids, from bacteriophage, from transposons, from yeast episomes, from insertion elements, from yeast chromosomal elements, from viruses, and vectors derived from combinations thereof, such as those derived from plasmid and bacteriophage genetic elements, such as cosmids and phagemids. The expression system constructs may contain control regions that regulate as well as engender expression. Generally, any system or vector suitable to maintain, propagate or express polynucleotides and to synthesize a polypeptide in a host may be used for expression in this regard. The appropriate DNA sequence may be inserted into the expression system by any of a variety of well-known and routine techniques, such as, for example, those set forth in Sambrook et al., supra.

The art is rich in patent and literature publications relating to “recombinant DNA” methodologies for the isolation, synthesis, purification and amplification of genetic materials for use in the transformation of selected host organisms. Thus, it is common knowledge to transform host organisms with “hybrid” viral or circular plasmid DNA which includes selected exogenous (i.e., foreign or “heterologous”) DNA sequences. The procedures known in the art first involve generation of a transformation vector by enzymatically cleaving circular viral or plasmid DNA to form linear DNA strands. Selected foreign DNA strands usually including sequences coding for desired protein product are prepared in linear form through use of the same/similar enzymes. The linear viral or plasmid DNA is incubated with the foreign DNA in the presence of ligating enzymes capable of effecting a restoration process and “hybrid” vectors are formed which include the selected exogenous DNA segment “spliced” into the viral or circular DNA plasmid.

### α-1,2-fucosyltransferase, α-1,3-fucosyltransferase

The genetically modified cell of the present invention comprises a heterologous, recombinant and/or synthetic nucleic acid which enables it to express an α-1,2-fucosyltransferase, and an α-1,3-fucosyltransferase.

Generally, and throughout the present disclosure, the term “glycosyltransferase activity” or “glycosyltransferase” designates and encompasses activity of enzymes that are responsible for the biosynthesis of disaccharides, oligosaccharides and polysaccharides. These enzymes catalyze the transfer of monosaccharide moieties from an activated nucleotide monosaccharide/sugar (the “glycosyl donor”) to a glycosyl acceptor molecule. The terms “alpha-1,2-fucosyltransferase” or “fucosyltransferase” or a nucleic acid/polynucleotide encoding an “alpha-1,2-fucosyltranferase” or “fucosyltransferase” refer to a glycosyltransferase that catalyzes the transfer of fucose from a donor substrate, for example, GDP-fucose, to an acceptor molecule in an alpha-1,2-linkage.

The terms “alpha-1,3-fucosyltranferase or fucosyltransferase” or a nucleic acid/polynucleotide encoding an “alpha-1,3-fucosyltranferase or fucosyltransferase” refer to a glycosyltransferase that catalyzes the transfer of fucose from a donor substrate for example, GDP-fucose, to an acceptor molecule in an alpha-1,3-linkage. The acceptor molecule can be, e.g., lactose, 2′-fucosyllactose, 3-fucosyllactose, or more complex HMO structures.

α-1,2-fucosyltransferases and α-1,3-fucosyltransferases are well-known in the art. Table 1 lists a non-limiting selection of α-1,2-fucosyltransferases and α-1,3-fucosyltransferases which may be encoded by the nucleic acid. Included in the present invention are also functional homologues of the α-1,2-fucosyltransferases and/or α-1,3-fucosyltransferases listed in table 1, which amino acid sequence is/are at least 80% identical, preferably at least 85% identical, more preferably at least 90%, such as 95%, 96%, 97%, 98% or 99% identical to the sequences given in in the respective protein sequence ID (GenBank).

TABLE 1
Fucosyltransferases

| | Protein Sequence ID | |
|---|---|---|
| Gene | (GenBank) | Description |
| futC | WP_080473865.1 | α-1,2-fucosyl-transferase |
| wbgL | ADN43847 | α-1,2-fucosyl-transferase |
| fucT2 | AAC99764 | α-1,2-fucosyl-transferase |
| MAMA_R764 | AGC02224.1 | α-1,3-fucosyl-transferase |
| Mg791 | AEQ33441.1 | α-1,3-fucosyl-transferase |
| Moumou_00703 | YP_007354660 | α-1,3-fucosyl-transferase |
| futA | WP_000487428.1 | α-1,3-fucosyl-transferase |
| fucT | AAB81031.1 | α-1,3-fucosyl-transferase |

In a presently preferred embodiment of a genetically modified cell according to the invention, the heterologous nucleic acid encoding an α-1,2-fucosyltransferase is a futC gene or a functional homologue thereof as defined above, such as the nucleotide sequence encoding the amino acid sequence of SEQ ID NO: 37 or an amino acid sequence with at least 90% identity, such as at least 95% identity to SEQ ID NO: 37. Normally, it would be expected that a genetically modified cell expressing a nucleic acid encoding an α-1,2-fucosyltransferase would primarily produce 2′FL.

In a presently preferred embodiment of a genetically modified cell according to the invention, the heterologous nucleic acid encoding an α-1,3-fucosyltransferase is selected from the group consisting of a futA gene, a fucT gene or a moumou gene and a functional homologue thereof as defined above.

In one embodiment, the heterologous α1,3-fucosyltransferase expressed comprises or preferably consists of a polypeptide that is identical with SEQ ID NO: 38. The protein according to SEQ ID NO:38 is a functional variant of FutA in which Ala (A) at position 128 is substituted by Asn (N) and His (H) at position 129 is substituted by Glu (E) (Choi et al. Biotechnol. Bioengin. 113, 1666 (2016)). A further functional variant of FutA is described in WO2020115671 in which Ala (A) at position 128 is substituted by Asn (N), His (H) at position 129 is substituted by Glu (E), Asp (D) at position 148 is substituted by Gly (G) and Tyr (Y) at position 221 is substituted by Cys (C). The protein according to SEQ ID No. 7 of WO2020115671 is termed FutA_mut2. In the present invention a further functional variant of FutA has been identified in which Ser (S) at position 46 is substituted with Phe (F), Ala (A) at position 128 is substituted by Asn (N), His (H) at position 129 is substituted by Glu (E), Tyr(Y) at position 132 is substituted with Ile(I), Asp (D) at position 148 is substituted by Gly (G) and Tyr (Y) at position 221 is substituted by Cys (C).

One embodiment of the present invention relates a 1,3-fuscosyltransferase with an amino acid sequence that is at least 90%, such as at least 95%, such as at least 98% identical to SEQ ID NO: 38 and which comprises or consists of the following substitutions S46F, A128N, H129E, Y1321, D148G and Y221C. In particular to the protein comprising or consisting of SEQ ID NO: 39, which is termed FutA_mut4 and which is encoded by the nucleotide sequence of SEQ ID NO: 32. The present invention further relates to the use of FutA_mut4 to produce DFL or 3FL.

In one embodiment the 1,3-fuscosyltransferase futA gene encodes an amino acid sequence comprising or consisting of the amino acid sequence of SEQ ID NO: 38 or SEQ ID NO: 39 or an amino acid sequence with at least 90% identity such as at least 95% identity to SEQ ID NO: 38 or SEQ ID NO: 39, such as the amino acid sequence of SEQ ID No. 7 of WO2020115671 (hereby incorporated by reference).

In another embodiment the 1,3-fuscosyltransferase fucT gene encodes an amino acid sequence comprising or consisting of the amino acid sequence of SEQ ID NO: 40 or an amino acid sequence with at least 90% identity such as at least 95% identity to SEQ ID NO: 40.

In another embodiment the 1,3-fuscosyltransferase moumou gene encodes an amino acid sequence comprising or consisting of the amino acid sequence of SEQ ID NO: 54 or an amino acid sequence with at least 90% identity such as at least 95% identity to SEQ ID NO: 54.

Normally, it would be expected that a genetically modified cell comprising a heterologous, recombinant and/or synthetic encoding for α-1,3 fucosyltransferase would primarily produce 3FL.

Therefore, it is surprising that the HMO produced in largest amounts by the genetically modified cell of the present invention is DFL. It is appreciated that the major amount of a single HMO is the DFL component.

Transporter Proteins

The invention provides recombinant cells capable of producing a human milk oligosaccharide (HMO), wherein the cells express an α-1,2-fucosyltransferase, an α-1,3-fucosyltransferase and a heterologous gene encoding a putative MFS (major facilitator superfamily) transporter protein. Said transporter gene typically originates from the bacterium Serratia marcescens, from the bacterium Rosenbergiella nectarea, or from the bacterium Pantoea vagans or from the bacterium Yersinia frederiksenii or from the bacterium Rouxiella badensis.

The major facilitator superfamily (MFS) transporter protein may be selected from, but is not limited to, marc, nec, vag, fred or bad. In certain embodiments the MFS transporter is not setA or YberC.

More specifically, the invention relates to a genetically modified cell optimized for the production of an oligosaccharide, in particular an HMO, wherein the heterologous, recombinant and/or synthetic nucleic acid encodes a transporter protein having at least 80% sequence identity, such as 90%, such as 95% sequence identity to an amino acid sequence of SEQ ID NO: 1 (MARC), or SEQ ID NO: 2 (NEC) or SEQ ID NO: 3 (VAG), or SEQ ID NO: 42 (FRED) or SEQ ID NO: 43 (BAD).

The amino acid sequence identified herein as SEQ ID NO: 1 is the amino acid sequence that is 100% identical to the amino acid sequence having the GenBank accession ID WP 060448169.1. The MFS transporter protein having the amino acid sequence of SEQ ID NO: 1 is identified herein as “Marc protein” or “Marc transporter” or “marc”, interchangeably; a nucleic acid sequence encoding marc protein is identified herein as “Marc coding nucleic acid/DNA” or “marc gene” or “marc”.

The amino acid sequence identified herein as SEQ ID NO: 2 is the amino acid sequence that is 100% identical to the amino acid sequence having the GenBank accession ID WP_092672081.1 (https://www.ncbi.nlm.nih.gov/protein/WP_092672081.1). The MFS transporter protein having the amino acid sequence of SEQ ID NO: 2 is identified herein as “Nec protein” or “Nec transporter” or “Nec”, interchangeably; a nucleic acid sequence encoding Nec protein is identified herein as “nec coding nucleic acid/DNA” or “nec gene” or “nec”.

The amino acid sequence identified herein as SEQ ID NO: 3 is the amino acid sequence that is 100% identical to the amino acid sequence having the GenBank accession ID WP_048785139.1 (https://www.ncbi.nlm.nih.gov/protein/WP_048785139.1). The MFS transporter protein having the amino acid sequence of SEQ ID NO: 3 is identified herein as “Vag protein” or “Vag transporter” or “Vag”, interchangeably; a nucleic acid sequence encoding Vag protein is identified herein as “vag coding nucleic acid/DNA” or “vag gene” or “vag”.

The amino acid sequence identified herein as SEQ ID NO: 42 is the amino acid sequence that is 100% identical to the amino acid sequence having the GenBank accession ID WP_087817556.1 (https://www.ncbi.nlm.nih.gov/protein/WP_087817556.1). The MFS transporter protein having the amino acid sequence of SEQ ID NO: 42 is identified herein as “Fred protein” or “Fred transporter” or “Fred”, interchangeably; a nucleic acid sequence encoding Fred protein is identified herein as “fred coding nucleic acid/DNA” or “fred gene” or “fred”.

The amino acid sequence identified herein as SEQ ID NO: 43 is the amino acid sequence that is 100% identical to the amino acid sequence having the GenBank accession ID WP_017489914.1 (https://www.ncbi.nlm.nih.gov/protein/WP_017489914.1). The MFS transporter protein having the amino acid sequence of SEQ ID NO: 43 is identified herein as “Bad protein” or “Bad transporter” or “Bad”, interchangeably; a nucleic acid sequence encoding Bad protein is identified herein as “bad coding nucleic acid/DNA” or “bad gene” or “bad”.

The invention relates to a genetically modified cell optimized for the production of one or more particular oligosaccharides, in particular one or more particular HMOs, such as fucosylated HMO's, such as 2′FL, 3FL, DFL or mixtures thereof, comprising a heterologous, recombinant and/or synthetic nucleic acid encoding a protein having at least 80% sequence identity, preferably at least 85%, more preferably at least 90%, and even more preferably at least 95% sequence identity, or even 100% sequence identity to the amino acid sequence of SEQ ID NO: 1 (MARC), or SEQ ID NO: 2 (NEC), or SEQ ID NO: 3 (VAG) or SEQ ID NO: 42 (FRED) or SEQ ID NO: 43 (BAD).

The putative MFS (major facilitator superfamily) transporter protein expressed in the genetically modified cell of the present invention preferably transports tri-HMOs and tetra-HMOs, e.g. trisaccharides such as 2′FL, 3FL and tetrasaccharides such as DFL.

Expression of a DNA sequence encoding a putative MFS (major facilitator superfamily) transporter protein, such as a Marc, Nec, Vag, Fred or Badprotein in the herein described HMO producing cells is found to be associated with an increase in total production of the HMOs, of which 50% w/w or more, such as 65% w/w or more produced by the cell are difucosyllactose (DFL).

Further, highly unexpectedly, expression of a putative MFS (major facilitator superfamily) transporter protein, such as a Marc protein, Nec or Vag or Fred or Bad protein in the herein described HMO producing cells leads to reduction in formation of the biomass during fermentation and to healthier cell cultures reflected by reduction in the number of dead cells at the end of fermentation, which makes the manufacturing process more efficient as more product is produced per biomass unit.

By the term “Major Facilitator Superfamily (MFS)” is meant a large and exceptionally diverse family of the secondary active transporter class, which is responsible for transporting a range of different substrates, including sugars, drugs, hydrophobic molecules, peptides, organic ions, etc. The specificity of sugar transporter proteins is highly unpredictable and the identification of novel transporter proteins with specificity towards for example oligosaccharides requires unburden laboratory experimentation (for more details see review by Reddy V. S. et al., (2012), FEBS J. 279(11): 2022-2035).

The term “MFS transporter” means in the present context a protein that facilitates transport of an oligosaccharide, preferably, an HMO, through the cell membrane, preferably transport of an HMO/oligosaccharide synthesized by the host cell from the cell cytosol to the cell medium, preferably an HMO/oligosaccharide comprising three or four sugar units, in particular, 2′FL and/or 3FL and/or DFL. Additionally, or alternatively, the MFS transporter, may also facilitate efflux of molecules which are not considered HMO or oligosaccharides according to the present invention, such as lactose, glucose, cell metabolites or toxins.

Genetic Modification of the Host Cell

To be able to synthesize one or more HMOs, the genetically modified host cell of the invention comprises at least one heterologous, recombinant and/or synthetic nucleic acid which encodes a functional enzyme with glycosyltransferase activity, comprising an α-1,2-fucosyltransferase and an α-1,3-fucosyltransferase which may be selected from the list given in Table 1. Preferably the glycosyltransferases are encoded by individual heterologous, recombinant and/or synthetic nucleic acids, such that at least two heterologous, recombinant and/or synthetic nucleic acids are present in the modified host cell to encode an α-1,2-fucosyltransferase and an α-1,3-fucosyltransferase.

The glycosyltransferase gene may be integrated into the genome (by chromosomal integration) of the host cell, or alternatively, it may be comprised in a plasmid DNA and expressed as plasmid-borne. The two or more heterologous, recombinant and/or synthetic nucleic acids encoding different enzymes with glycosyltransferase activity may be integrated in the genome and/or expressed from a plasmid, e.g. an α-1,2-fucosyltransferase (a first heterologous, recombinant and/or synthetic nucleic acid encoding a first glycosyltransferase) in combination with an α-1,3-fucosyltransferase (a second heterologous, recombinant and/or synthetic nucleic acid encoding a second glycosyltransferase), where the first and second heterologous, recombinant and/or synthetic nucleic acid can independently from each other be integrated chromosomally or on a plasmid. In one preferred embodiment, both the first and second heterologous, recombinant and/or synthetic nucleic acids are stably integrated into the chromosome of the production cell; in another embodiment at least one of the first and second glycosyltransferase is plasmid-borne.

In addition, the putative MFS (major facilitator superfamily) transporter protein gene may be integrated into the genome (by chromosomal integration) of the host cell, or alternatively, it may be comprised in a plasmid DNA and expressed as plasmid-borne. The first and second and further heterologous, recombinant and/or synthetic nucleic acid can independently from each other be integrated chromosomally or on a plasmid. In one preferred embodiment, the first, second and further heterologous, recombinant and/or synthetic nucleic acids are stably integrated into the chromosome of the production cell; in another embodiment at least one of the first, second and further heterologous, recombinant and/or synthetic nucleic acid is plasmid-borne.

The heterologous, recombinant and/or synthetic nucleic acid sequence of the invention may be a coding DNA sequence, e.g. a gene, or non-coding DNA sequence, e.g. a regulatory DNA, such as a promoter sequence. One aspect of the invention relates to providing a recombinant cell comprising recombinant DNA sequences encoding enzymes necessary for the production of one or more HMOs and a DNA sequence encoding a sugar transporter protein. Accordingly, in one embodiment the invention relates to a nucleic acid construct comprising a coding nucleic sequence, i.e. a heterologous, recombinant and/or synthetic DNA sequence of a gene of interest, e.g. a glycosyltransferase gene or a MFS gene, and a non-coding DNA sequence, e.g. a promoter DNA sequence, e.g. a recombinant promoter sequence derived from the promoter of lac operon, an mglB operon or an glp operon, or a promoter sequence derived from another genomic promoter DNA sequence, or a synthetic promoter sequence, wherein the coding and promoter sequences are operably linked.

In one embodiment, the nucleic acid construct of the invention may be a part of the vector DNA, in another embodiment the construct it is an expression cassette/cartridge that is integrated in the genome of a host cell. Accordingly, the term “nucleic acid construct” means an artificially constructed segment of nucleic acid, in particular a DNA segment, which is intended to be ‘transplanted’ into a target cell, e.g. a bacterial cell, to modify expression of a gene of the genome or express a gene/coding DNA sequence which may be included in the construct. In the context of the invention, the nucleic acid construct contains a recombinant DNA sequence comprising two or more recombinant DNA sequences: essentially, a non-coding DNA sequence comprising a promoter DNA sequence and a coding DNA sequence encoding a gene of interest, e.g. sugar transporter protein, a glycosyltransferase, and/or another gene useful for production of an HMO in a host cell.

Preferably, the construct comprises further non-coding DNA sequences that either regulate transcription or translation of the coding DNA of the construct, e.g. a DNA sequence facilitating ribosome binding to the transcript, a leading DNA sequence that stabilize the transcript.

Integration of the recombinant gene of interest comprised in the construct (expression cassette) into the bacterial genome can be achieved by conventional methods, e.g. by using linear cartridges that contain flanking sequences homologous to a specific site on the chromosome, as described for the attTn7-site (Waddell C. S. and Craig N. L., Genes Dev. (1988) Feb; 2(2):137-49); methods for genomic integration of nucleic acid sequences in which recombination is mediated by the Red recombinase function of the phage λ or the RecE/RecT recombinase function of the Rac prophage (Murphy, J Bacteriol. (1998); 180(8):2063-7; Zhang et al., Nature Genetics (1998) 20: 123-128 Muyrers et al., EMBO Rep. (2000) 1(3): 239-243); methods based on Red/ET recombination (Wenzel et al., Chem Biol. (2005), 12(3):349-56; Vetcher et al., Appl Environ Microbiol. (2005); 71(4):1829-35); or positive clones, i.e. clones that carry the expression cassette, can be selected e.g. by means of a marker gene, or loss or gain of gene function.

A single copy of the expression cassette comprising a gene of interest may be sufficient to secure production of the desired HMO and achieve the desired effects according to the invention. Accordingly, in some preferred embodiments, the invention relates to a recombinant HMO producing cell that comprises one, two or three copies of a gene of interest integrated in the genomic DNA of the cell. In some embodiments the single copy of the gene is preferred.

In one preferred embodiment, recombinant coding nucleic acid sequence of the nucleic acid construct of the invention is heterologous with respect to the promoter, which means that in the equivale native coding sequence in the genome of species of origin is transcribed under control of another promoter sequence (i.e. not the promoter sequence of the construct). Still, with respect to the host cell, the coding DNA may be either heterologous (i.e. derived from another biological species or genus), such as e.g. the DNA sequence encoding a sugar transporter protein expressed in Escherichia coli host cells, or homologous (i.e. derived from the host cell), such as e.g. genes of the colonic acid operon, e.g., the gmd, wcaG, manC, manB genes also disclosed as SEQ ID NO: 30 herein.

The term, a “regulatory element” or “promoter” or “promoter region” or “promoter element” is a nucleic acid sequence that is recognized and bound by a DNA dependent RNA polymerase during initiation of transcription and provide a site for initiation of the transcription into mRNA. The promoter, together with other transcriptional and translational regulatory nucleic acid sequences (also termed “control sequences”) is necessary to express a given gene or group of genes (an operon) by biding proteins that determine the frequency (or rate) of transcriptional initiation including transcription inhibition. Promoter elements and most regulatory elements are usually “upstream” of (i.e., preceding) the gene to be transcribed. DNA sequences which are downstream of the encoding gene can provide a signal for termination of the transcription into mRNA are referred to as transcription “terminator” sequences. In general, the transcriptional and translational regulatory sequences include, but are not limited to, promoter sequences, ribosomal binding sites, transcriptional start and stop sequences, translational start and stop sequences, and enhancer or activator sequences. The “transcription start site” means the first nucleotide to be transcribed and is designated +1. Nucleotides downstream of the start site are numbered +2, +3, +4 etc., and nucleotides in the 5′ opposite (upstream) direction are numbered −1, −2, −3 etc. The promoter DNA sequence of the construct can derive from a promoter region of any gene of the genome of a selected species, preferably, a promoter region of the genomic DNA of E. coll. Accordingly, any promoter DNA sequence that is able to bind to an RNA polymerase and initiate transcription is suitable for practicing the invention. In principle, any promoter DNA sequence can be used to control transcription of the heterologous, recombinant and/or synthetic gene of interest of the construct, different or same promoter sequences may be used to drive transcription of different genes of interest integrated in the genome of the host cell or in expression vector DNA. To have an optimal expression of the heterologous, recombinant and/or synthetic genes included in the construct, the construct may comprise further regulatory sequences, e.g. a leading DNA sequence, such as a DNA sequence derived from 5′-untranslated region (5′UTR) of a glp gene of E. coli, a sequence for ribosomal binding. Examples of the later sequences are described in WO2019123324 and WO2020255054 (incorporated herein by reference).

In some preferred embodiments, the regulatory element for the regulation of the expression of a recombinant gene included in the construct of the invention is glpFKX operon promoter, PglpF, in other preferred embodiments, the promoter is lac operon promoter, Plac. However, any promoter enabling transcription and/or regulation of the level of transcription of one or more heterologous, recombinant and/or synthetic s that encode one or more proteins (or one or more regulatory nucleic acids) that are either necessary or beneficial to achieve an optimal level of biosynthetic production of one or more HMOs in the host cell, e.g. proteins involved in transmembrane transport of HMO, or HMO precursors, degradation of by-products of the HMO production, gene expression regulatory proteins, etc, and allowing to achieve the desired effects according to the invention is suitable for practicing the invention.

A fucosyltransferase gene and/or a sugar transporter gene according to the present invention can also be operably linked to a PglpF promoter element and be expressed from the corresponding genome-integrated cassette, it can be expressed under the control of a glp promoter, mglB promoter, or under the control of any other promoter suitable for the expression system, e.g. Plac.

In a presently preferred aspect, a fucosyltransferase gene and/or a MFS transporter gene according to the present invention is operably linked to a PmglB-promoter and is expressed from the corresponding genome-integrated cassette. In particular, said promotor can be PmglB_70 UTR_SD4 as shown in SEQ ID NO:4.

Preferably, the construct of the invention comprising a gene related to biosynthetic production of an HMO, a promoter DNA sequence, and other regulatory sequences, such as a ribosomal binding site sequence (e.g. Shine-Dalgarno sequence), expressed in the host cell enables production of the HMO at the level of at least 0.03 g/OD (optical density) of 1 liter of the fermentation media comprising a suspension of host cells, e.g., at the level of around 0.05 g/l/OD to around 0.1 g/l/OD. For the purposes of the invention, the later level of HMO production is regarded as “sufficient” and the host cell capable of producing this level of a desired HMO is regarded as “suitable host cell”, i.e. the cell can be further modified to express the MFS transporter protein, e.g. Marc, or Nec, or Vag, or Fred or Bad to achieve at least one effect described herein that is advantageous for the HMO production.

Genetically modified cells of the invention can be provided using standard methods of the art e.g. those described in the manuals by Sambrook et al., Wilson & Walker, Maniatise et al, and Ausubel et al.

A host cell suitable for the HMO production, e.g. E. coli, may comprise an endogenous β-galactosidase gene or an exogenous β-galactosidase gene, e.g. E. coli comprises an endogenous lacZ gene (e.g., GenBank Accession Number V00296 (GI:41901)). For the purposes of the invention, an HMO-producing cell is genetically manipulated to comprise the gene that is inactivated. The lacZ gene may be inactivated by a complete or partial deletion of the corresponding nucleic acid sequence from the bacterial genome, or the gene sequence is mutated in the way that it is transcribed, or, if transcribed, the transcript is not translated or if translated to a protein (i.e. β-galactosidase), the protein does not have the corresponding enzymatic activity. In this way the HMO-producing bacterium accumulates an increased intracellular lactose pool which is beneficial for the production of HMOs.

Method for the Production of One or More HMOs

A further aspect of the invention relates to a method for the production of one or more oligosaccharides, wherein 45%, such as 50% w/w, such as 65% w/w, or more of the HMOs produced in the cell is difucosyllactose (DFL), the method comprising the steps of:
(i) providing a genetically modified cell capable of producing an HMO, wherein said cell comprises a heterologous, recombinant and/or synthetic nucleic acid encoding
   a. an α-1,2-fucosyltransferase, and
   b. an α-1,3-fucosyltransferase, and
(ii) culturing the cell according to (i) in a suitable cell culture medium to produce said HMO; and
(iii) harvesting one or more HMOs produced in step (ii).

In a presently preferred aspect, said genetically modified cell further comprises a heterologous, recombinant and/or synthetic nucleic acid further encodes a transporter protein selected from the major facilitator superfamily (MFS).

Preferably the method using the genetically modified cell comprising a recombinant MFS transporter protein produces at least 5% w/w more DFL compared to the same method wherein the genetically modified cell is not expressing the recombinant MFS transporter protein.

In particular, the method according to the present invention facilitates that at the most 45% w/w of the total amount of the HMOs produced in the cell is 3-fucosyllactose (3FL), 2′-fucosyllactose (2′FL) and/or lactose, such as at the most 30% w/w of the total amount of the HMOs produced in the cell.

The method of the present invention is herein demonstrated to result primarily in the production of difucosyllactose (DFL) with a relatively low content of 3-fucosyllactose (3FL) and/or 2′-fucosyllactose (2′FL).

Depending on the fermentation condition and the expression level of the enzymes, method of the present invention results mainly in the production of difucosyllactose (DFL) with a relatively low content of 2′-fucosyllactose (2′FL) and less than 1% w/w of the total HMOs 3-fucosyllactose (3FL).

Thus, in one aspect of the present invention, the method of the present invention results in the production of DFL (at least 60% w/w, such as at least 65% w/w, such as at least 70% w/w of the total HMOs), 2′FL (no more than 35% w/w, such as less than 30% w/w of the total HMOs), and surprisingly essentially no 3FL.

Depending on the fermentation condition and the expression level of the enzymes, the method of the present invention results mainly in the production of difucosyllactose (DFL) with a relatively low content of 3-fucosyllactose (3FL) and less than 1% w/w of the total HMOs of 2′-fucosyllactose (2′FL).

Thus, in one aspect of the present invention, the method of the present invention results in the production of DFL (at least 60% w/w, such as at least 65% w/w, such as at least 70% w/w of the total HMOs), 3FL (no more than 35% w/w, such as less than 30% w/w of the total HMOs), and surprisingly essentially no 2′FL.

In addition, as disclosed in the experimental section, the method of the present invention including the expression of a transporter protein selected from the major facilitator superfamily (MFS) enhances the selective production of DFL even further, such as with up to 25%, such as with 5%, 10% 15%, 20% or 25% w/w of the total HMOs.

Thus, in one aspect, the method of the present invention results in the production of DFL (at least 55% w/w, 60% w/w, such as at least, such as at least 65% w/w of the total HMOs) and 2′FL, 3FL (no more than 45% w/w, such as no more than 35% of the total HMOs).

Thus, in one aspect, the method of the present invention results in the production of DFL (at least 65% w/w of the total HMOs) and 2′FL, 3FL (no more than 35% w/w of the total HMOs).

Thus, in one aspect, the method of the present invention results in the production of DFL (at least 70% w/w of the total HMOs) and 2′FL, 3FL (no more than 30% w/w of the total HMOs).

In a particularly preferred aspect, the in one aspect, the method of the present invention results in the production of DFL (at least 90% w/w of the total HMOs) and 2′FL, 3FL (no more than 10% w/w of the total HMOs).

Consequently, the present invention relates to a method of producing one or more Human Milk Oligosaccharides (HMOs), wherein 50% w/w, such as 65% w/w or more of the HMOs produced by the cell are difucosyllactose (DFL).

In a presently preferred aspect, the present invention relates to method, wherein 55% w/w or more of the HMOs produced by the cell are difucosyllactose (DFL).

In one aspect of the invention, the method of the invention produces at least 50% w/w of the total HMOs, such as between 50-99%, such as at least 55, 60, 65, 70, 75, 80, 85, 90, 95 or 99% DFL. Preferably 70, 75, 80, 85, 90, 95 or 99% DFL.

In one aspect of the invention, the method of the present invention produces at the most 45% w/w of the total HMOs, such as between 5-10, 5-15, 5-30, 10-30%, such as at the most 0.5, 1, 5, 10, 5, 20, 25, 30, 35% 2′FL and/or 3FL.

In one aspect of the invention, the method of the present invention produces at the most 5% w/w of the total HMOs, such as between 0-5%, such as at the most 0.1, 0.2, 0.3, 0.4, 0.5, 0.6, 0.7, 0.8, 0.9, 1, 1.5, 2, 2.5, 3, 3.5, 4, 4.5 or 5% 2′FL and/or 3FL.

A method according to the present invention includes lactose. The amount of lactose in the fermentation is dependent on the fermentation conditions and the expression level as well as the choice of enzymes and the optional sugar transporter protein expressed. The combined amount of produced 2′FL, 3FL together with lactose will not be more than 49%, such as 45% w/w such as less than 35% w/w of the total oligosaccharides produced by the cell and/or the method described herein.

The currently disclosed method comprises (i) providing a genetically modified cell capable of producing an HMO, wherein said cell comprises a heterologous, recombinant and/or synthetic nucleic acid encoding an α-1,2-fucosyltransferase and an α-1,3-fucosyltransferase, and optionally a transporter protein selected from the major facilitator superfamily (MFS), such as but not limited to a protein of SEQ ID NO: 1 or 2 or 3 or 42 or 43, or a functional homologue thereof which amino acid sequence is at least 80% identical, preferably at least 85% identical, more preferably at least 90% identical to SEQ ID NO: 1 or 2 or 3 or 42 or 43; (ii) culturing the cell of (i) in a suitable cell culture medium and (iii) harvesting the HMO(s) produced in step (ii).

To produce one or more HMOs, the HMO-producing bacteria as described herein are cultivated according to the procedures known in the art in the presence of a suitable carbon source. Typically, carbon source(s) is/are selected from the group consisting of glycerol, glucose, sucrose and mixtures thereof. Alternative carbon sources can be selected from molasses, corn syrup, galactose, succinate, malate, pyruvate, lactate, ethanol, methanol, citrate and raffinose. The produced HMO is harvested from the cultivation media and the microbial biomass formed during the cultivation process. Thereafter, the HMOs are purified according to the procedures known in the art, e.g. such as described in WO2015188834, WO2017182965 or WO2017152918, and the purified HMOs are used as nutraceuticals, pharmaceuticals, or for any other purpose, e.g. for research.

Manufacturing of HMOs is typically accomplished by performing cultivation in larger volumes. The term “manufacturing” and “manufacturing scale” in the meaning of the invention defines a fermentation with a minimum volume of 100 L, such as 1000 L, such as 10.000 L, such as 100.000 L, such as 200.000 L culture broth. Usually, a “manufacturing scale” process is defined by being capable of processing large volumes of a preparation an HMO(s) of interest and yielding amounts of the HMO of interest that meet, the demands for toxicity tests, clinical trials as well as for market supply. In addition to the large volume, a manufacturing scale method, as opposed to simple lab scale methods like shake flask cultivation, is characterized by the use of the technical system of a bioreactor (fermenter) which is equipped with devices for agitation, aeration, nutrient feeding, monitoring and control of process parameters (pH, temperature, dissolved oxygen tension, back pressure, etc.). To a large extent, the behaviour of an expression system in a lab scale method, such as shake flasks, benchtop bioreactors or the deep well format described in the examples of the disclosure, does allow to predict the behaviour of that system in the complex environment of a bioreactor.

With regard to the suitable cell cultivation medium used in the fermentation process, there are no limitations. The culture medium may be semi-defined, i.e. containing complex media compounds (e.g. yeast extract, soy peptone, casamino acids, etc.), or it may be chemically defined, without any complex compounds.

In one aspect, the method described herein comprises culturing of the cell in step (ii) which is conducted at low lactose conditions. In the present context, low lactose conditions are typically considered to be conditions having less than 5 g lactose/l culture medium, such as less than 4 g lactose/l culture medium, less than 3 g lactose/l culture medium, less than 2 g lactose/l culture medium, less than 1 g lactose/l culture medium. In one particular aspect, the culturing of the cell in step (ii) is conducted at essentially lactose-free conditions, or at least at conditions with no addition of lactose to the culturing medium other than what is produced by the genetically modified cell itself.

The term “harvesting” in the context of the invention relates to collecting the produced HMO(s) following the termination of fermentation. In different embodiments it may include collecting the HMO(s) included in both the biomass (i.e. inside the host cells) and cultivation media (supernatant/fermentation broth), i.e. before/without separation of the fermentation broth from the biomass. In other embodiments the produced HMOs may be collected separately from the biomass and fermentation broth, i.e. after/following the separation of biomass from cultivation media (i.e. fermentation broth). The separation of cells from the medium can be carried out with any of the methods well known to the skilled person in the art, such as any suitable type of centrifugation or filtration. The separation of cells from the medium can follow immediately after harvesting the fermentation broth or be carried out at a later stage after storing the fermentation broth at appropriate conditions. Recovery of the produced HMO(s) from the remaining biomass (or total fermentation) include extraction thereof from the biomass (i.e the production cells). It can be done by any suitable methods of the art, e.g. by sonication, boiling, homogenization, enzymatic lysis using lysozyme, or freezing and grinding.

After recovery from fermentation, HMO(s) are available for further processing and purification.

The HMOs produced by recombinant cells of the invention may be purified using a suitable procedure available in the art, e.g. as illustrated in FIG. 7 or as described in WO2016095924, WO2015188834, WO2017152918, WO2017182965, WO2017152918, or US20190119314 (all incorporated by reference).

Cells and methods for HMO production described herein allow for controlled production of an HMO product with a defined HMO profile, e.g. in the produced HMO mixture, DFL is the dominating HMO (product) compared to the other HMOs i.e. 3FL and 2′FL (by-products) of the mixture. Thus, DFL is produced in substantially higher amounts than the other by-product HMOs (3FL and/or 2′FL). With the genetically modified cells of the present invention the level of 3FL and/or 2′FL in the DFL product can be significantly reduced.

Advantageously, the invention provides both a decreased ratio of by-product to product, i.e. decreased ratio of 2′FL/3FL/DFL, and an increased overall yield of the total HMOs (and/or HMOs in total). The reduced by-product formation in relation to product formation facilitates an elevated product formation and increases efficiency of both the production and product recovery process, providing superior manufacturing procedure of HMOs and in particular for DFL production.

In one preferred embodiment, the product is DFL and the by-product is 3FL. In another preferred embodiment, the product is DFL and the by-product is 2′FL. In another preferred embodiment, the product is DFL, and the by-products are 3FL and 2′FL.

The invention is further illustrated by non-limiting examples and embodiments below.

### BRIEF DESCRIPTION OF THE FIGURES

FIG. 1

Relative production of 2′FL, 3FL, and DFL, in modified E. coli strains producing 2′FL, 3FL, or DFL, respectively. The modified E. coli DFL strain overexpresses the α-1,2-fucosyltransferase gene, futC and the α-1,3-fucosyltransferase gene, futA. The HMO levels are given relatively to the 2′FL produced by strain 1. Data is obtained from deep-well fed-batch assay.

FIG. 2

Relative production of total HMO in a modified E. coli DFL production strain overexpressing the homologous sugar efflux transporter A gene (setA) in strain 4, or the heterologous MFS transporter genes marc, nec, or vag, in strain 5-7, respectively. The HMO levels are shown relatively to the total HMO produced in strain 3. Data is obtained from deep-well fed-batch assay.

FIG. 3

Relative distribution of 2′FL and DFL in a modified E. coli DFL production strain overexpressing the homologous sugar efflux transporter A gene (setA) in strain 4, or the heterologous MFS transporter genes marc, nec, or vag, in strain 5-7, respectively. The relative ratio of DFL and 2′FL are shown relatively to the total amount HMO produced by each strain. Data is obtained from deep-well fed-batch assay.

FIG. 4

Time profiles for the lactose monohydrate concentration in the fermentation broth throughout the two runs at either high lactose (process 1, solid line) or low lactose (process, dotted line) condition using the DFL producing strain 8.

FIG. 5

Time profiles of the ratio DFL/(2′FL+DFL) in % by mass in the fermentation broth throughout the two runs at either high lactose condition (process 1, solid line) or low lactose condition (process 2, dotted line) using strain 8. 3FL is in all cases below1% of the total sum of HMO and therefore negligible.

FIG. 6

Time profiles of the relative formation of DFL titer in the fermentation broth throughout the two runs at either high lactose condition (process 1, solid line) or low lactose condition (process 2, dotted line) using strain 8. The DFL titer is shown relative to the end point measurement of strain 8 process 2 (low lactose level).

FIG. 7

Purification steps of the fermentation broth to obtain crystalline DFL. Ultrafiltration (UF) is used to separate biomass from the broth, nanofiltration (NF) to concentrate the broth, ion exchange resin (IEX) to remove salts and activated charcoal (AC) to remove color. Selective DFL crystallization as the final step provides DFL in very high purity.

### ITEMS

1. A genetically modified cell capable of producing one or more Human Milk Oligosaccharides (HMOs), wherein said cell comprises a heterologous, recombinant and/or synthetic nucleic acid encoding
a. an α-1,2-fucosyltransferase, and
b. an α-1,3-fucosyltransferase,
   wherein 50% w/w or more, such as more than 60% of the HMOs produced by the cell are difucosyllactose (DFL).

2. The genetically modified cell according to item 1, wherein the cell further comprises
c. a heterologous, recombinant and/or synthetic nucleic acid encoding a transporter protein selected from the major facilitator superfamily (MFS).

3. The genetically modified cell according to items 1 or 2, wherein the MFS transporter protein originates from a bacterium selected from the group consisting of Serratia marcescens, Rosenbergiella nectarea, Pantoea vagans, Yersinia frederiksenii and Rouxiella badensis.

4. The genetically modified cell according to any one of the preceding items, wherein the transporter protein is selected from the group consisting of SEQ ID NO: 1 (Marc), SEQ ID NO: 2 (Nec), SEQ ID NO: 3 (Vag), SEQ ID NO: 42 (fred) and SEQ ID NO: 43 (bad) or a functional homologue thereof which amino acid sequence is at least 80%, such as at least 85% or at least 90% identical to SEQ ID NO: 1 (Marc), SEQ ID NO: 2 (Nec), SEQ ID NO: 3 (Vag), SEQ ID NO: 42 (fred) or SEQ ID NO: 43 (bad).

5. The genetically modified cell according to any one of the preceding items, wherein the genetically modified cell with the MFS transporter protein produces at least 5% w/w more DFL compared to the same cell without the MFS transporter protein.

6. The genetically modified cell according to any one of the preceding items, wherein 65%, such as 70% w/w or more of the HMOs produced by the cell are difucosyllactose (DFL).

7. The genetically modified cell according to any one of any one of the preceding items, wherein said heterologous, recombinant and/or synthetic nucleic acid encoding an α-1,2-fucosyltransferase is a futC gene or a wbgL A gene, or a functional homologue thereof.

8. The genetically modified cell according to item 7, wherein the futC gene encodes an amino acid sequence comprising or consisting of the amino acid sequence of SEQ ID NO: 37 or a functional homologue thereof which is at least 90% identical to the amino acid sequence of SEQ ID NO: 37 and the wbgL gene comprises or consists of the amino acid sequence of NCBI accession nr ADN43847, or a functional homologue thereof which is at least 90% identical to the amino acid sequence of NCBI accession nr ADN43847.

9. The genetically modified cell according to any one of the preceding items, wherein said heterologous, recombinant and/or synthetic nucleic acid encoding an α-1,3-fucosyltransferase is a futA gene or a fucT gene or moumou gene, or a functional homologue thereof.

10. The genetically modified cell according to item 9, wherein the futA gene encodes an amino acid sequence comprising or consisting the amino acid sequence of SEQ ID NO: 38 or SEQ ID NO: 39 or a functional homologue thereof which is at least 90% identical to the amino acid sequence of SEQ ID NO: 38 or SEQ ID NO: 39 and the fucT gene encodes an amino acid sequence comprising or consisting the amino acid sequence of SEQ ID NO: 40, or a functional homologue thereof which is at least 90% identical to the amino acid sequence of SEQ ID NO: 40 and the moumou gene encodes an amino acid sequence comprising or consisting the amino acid sequence of SEQ ID NO: 54, or a functional homologue thereof which is at least 90% identical to the amino acid sequence of SEQ ID NO: 54.

11. The genetically modified cell according to any one of the preceding items, wherein the heterologous, recombinant and/or synthetic nucleic acid encoding an α-1,3-fucosyltransferase is the fucT gene encoding an amino acid sequence of SEQ ID NO: 40, or a functional homologue thereof which is at least 90% identical to the amino acid sequence of SEQ ID NO: 40.

12. The genetically modified cell according to any one of the preceding items, wherein the heterologous, recombinant and/or synthetic nucleic acid encoding an α-1,3-fucosyltransferase is the futA gene encoding an amino acid sequence of SEQ ID NO: 38 or SEQ ID NO: 39 or a functional homologue thereof which is at least 90% identical to the amino acid sequence of SEQ ID NO: 38 or SEQ ID NO: 39.

13. The genetically modified cell according to any one of the preceding items the ratio of the active fucosyltransfeases, α-1,2-fucosyltransferase to α-1,3-fucosyltransferase is in the range from 1:1 to 2:5, such as 1:1, 1:2, 1:3; 1:4, 1:5, 2:3 or 2:5.

14. The genetically modified cell according to item 13, where in the FutC:FutA ratio is 1:3 or 2:3.

15. The genetically modified cell according to item 12, wherein the cell further comprises a heterologous, recombinant and/or synthetic nucleic acid encoding the α-1,2-fucosyltransferase FutC and a heterologous, recombinant and/or synthetic nucleic acid encoding a MFS transporter or a functional homologue thereof selected from Item 4.

16. The genetically modified cell according to item 11, wherein the cell further comprises a heterologous, recombinant and/or synthetic nucleic acid encoding the α-1,2-fucosyltransferase FutC and a heterologous, recombinant and/or synthetic nucleic acid encoding a nec or marc MFS transporter or a functional homologue thereof from Item 4.

17. The genetically modified cell according to any one of the preceding items, wherein at the most 45%, such as at most 35%, w/w of the total amount of the HMOs produced in the cell is 3-fucosyllactose (3FL), or 2′-fucosyllactose (2′FL).

18. The genetically modified cell according to any one of the preceding items, wherein at the most 30% w/w, such as at the most 20% w/w, at the most 15% w/w, at the most 10% w/w, at the most 5% w/w, at the most 2.5% w/w, or at the most 1% w/w of the total amount of the HMOs produced in the cell is 3-fucosyllactose (3FL).

19. The genetically modified cell according to any one of the preceding items, wherein at the most 30% w/w, such as at the most 20% w/w, at the most 15% w/w, at the most 10% w/w, at the most 5% w/w, at the most 2.5% w/w, or at the most 1% w/w of the total amount of the HMOs produced in the cell is 2′-fucosyllactose (2′FL).

20. The genetically modified cell according to any one of the preceding items, wherein the genetically modified cell is a microbial cell.

21. The genetically modified cell according to any one of the preceding items, wherein the genetically modified cell is Escherichia coli.

22. The genetically modified cell according to any one of the preceding items, wherein the cell further comprises a heterologous, recombinant and/or synthetic regulatory element comprising a nucleic sequence for the regulation of the expression of the heterologous, recombinant and/or synthetic nucleic acid.

23. The genetically modified cell according to item 22, wherein the regulatory element for the regulation of the expression of the heterologous, recombinant and/or synthetic nucleic acid comprises a promoter nucleic sequence such as a lac promoter, Plac, or a mglB promoter, PmglB, or a glp promoter, PglpF, or any variation thereof.

24. The genetically modified cell according to item 23, wherein the regulatory element for the regulation of the expression of the α-1,2-fucosyltransferase in the heterologous, recombinant and/or synthetic nucleic acid comprises a promoter nucleic sequence which is PglpF or a variant thereof.

25. The genetically modified cell according to item 24, wherein the PglpF promoter comprises or consists of the nucleic acid sequence of SEQ ID NO: 29 or a nucleic acid sequence which is at least 90%, such as 95% identical to SEQ ID NO: 29.

26. The genetically modified cell according to item 22 or 23, wherein the regulatory element for the regulation of the expression of the α-1,3-fucosyltransferase in the heterologous, recombinant and/or synthetic nucleic acid comprises a promoter nucleic sequence which is PmglB or a variant thereof.

27. The genetically modified cell according to item 26, wherein the PmglB promoter is a variant which comprises or consists of the nucleic acid sequence of SEQ ID NO: 4 or a nucleic acid sequence which is at least 90%, such as 95% identical to SEQ ID NO: 4.

28. A method for the production of one or more oligosaccharides, wherein 50% w/w, such as 70% w/w or more of the HMOs produced in the cell is difucosyllactose (DFL), the method comprising the steps of:
(i) providing a genetically modified cell capable of producing an HMO, wherein said cell comprises a heterologous, recombinant and/or synthetic nucleic acid encoding
   a. an α-1,2-fucosyltransferase, and
   b. an α-1,3-fucosyltransferase, and
(ii) culturing the cell according to (i) in a suitable cell culture medium to produce said HMO; and
(iii) harvesting one or more HMOs produced in step (ii).

29. The method according to item 28, wherein said cell further comprises a heterologous, recombinant and/or synthetic nucleic acid encoding a transporter protein selected from the major facilitator superfamily (MFS).

30. The method according to Item 29, wherein the genetically modified cell with a heterologous, recombinant and/or synthetic nucleic acid encoding a MFS transporter protein produces at least 5% w/w more DFL compared to the same cell without the MFS transporter protein.

31. The method according to item 28 to 30, wherein 65%, such as 70% w/w or more of the HMOs produced by the cell is difucosyllactose (DFL).

32. The method according to item 28 to 31, wherein said heterologous, recombinant and/or synthetic nucleic acid encoding an α-1,2-fucosyltransferase is a futC gene or a wbgL gene, or a functional homologue thereof.

33. The method according to item 32, wherein the futC gene encodes an amino acid sequence comprising or consisting of the amino acid sequence of SEQ ID NO: 37 or a functional homologue thereof which is at least 90% identical to the amino acid sequence of SEQ ID NO: 37 and the wbgL gene encodes an amino acid sequence comprising or consisting of the amino acid sequence of NCBI accession nr ADN43847, or a functional homologue thereof which is at least 90% identical to the amino acid sequence of NCBI accession nr ADN43847.

34. The method according to item 28 to 33, wherein said heterologous, recombinant and/or synthetic nucleic acid encoding an α-1,3-fucosyltransferase is a futA gene or a fucT gene or moumou gene, or a functional homologue thereof.

35. The method according to item 34, wherein the futA gene encodes an amino acid sequence comprising or consisting of the amino acid sequence of SEQ ID NO: 38 or SEQ ID NO: 39 or a functional homologue thereof which is at least 90% identical to the amino acid sequence of SEQ ID NO: 38 or SEQ ID NO: 39 and the fucT gene encodes an amino acid sequence comprising or consisting of the amino acid sequence of SEQ ID NO: 40, or a functional homologue thereof which is at least 90% identical to the amino acid sequence of SEQ ID NO: 40 and the moumou gene encodes an amino acid sequence comprising or consisting of the amino acid sequence of SEQ ID NO: 54, or a functional homologue thereof which is at least 90% identical to the amino acid sequence of SEQ ID NO: 54.

36. The method according to any one of items 28 to 35, wherein at the most 30% w/w of the total amount of the HMOs produced in the cell is 3-fucosyllactose (3FL) or 2′-fucosyllactose (2′FL).

37. The method according to any one of items 28 to 36, wherein at the most 30%, such as at the most 20% w/w, at the most 15% w/w, at the most 10% w/w, at the most 5% w/w, at the most 2.5% w/w, or at the most 1% w/w of the total amount of the HMOs produced in the cell is 3-fucosyllactose (3FL).

38. The method according to any one of items 28 to 37, wherein at the most 30%, such as at the most 20% w/w, at the most 15% w/w, at the most 10% w/w, at the most 5% w/w, at the most 2.5% w/w, or at the most 1% w/w of the total amount of the HMOs produced in the cell is 2′-fucosyllactose (2′FL).

39. The method according to anyone of items 28 to 38, wherein the culturing of the cell in step (ii) is conducted at low lactose conditions.

40. The method according to item 39, wherein the culturing of the cell in step (ii) is conducted at conditions having <5 g lactose/l culture medium.

41. Use of a genetically modified cell according to any one of items 1 to 27 for the production of one or more HMO, wherein at least 65% w/w, such as 70% w/w or more of the HMOs produced in the cell is difucosyllactose (DFL).

42. A 1,3-fuscosyl transferase with an amino acid sequence that is at least 90%, such as at least 95%, such as at least 98% identical to SEQ ID NO: 38 and which comprises or consists of the following substitutions S46F, A128N, H129E, Y1321, D148G and Y221C.

43. The 1,3-fuscosyl transferase according to item 42, wherein the amino acid sequence comprises or consist of SEQ ID NO: 39.

44. The 1,3-fuscosyl transferase according to item 42 or 43, wherein the 1,3-fuscosyl transferase is encoded by the nucleotide sequence of SEQ ID NO: 32.

### EXAMPLES

Materials and Methods

Unless otherwise noted, standard techniques, vectors, control sequence elements, and other expression system elements known in the field of molecular biology are used for nucleic acid manipulation, transformation, and expression. Such standard techniques, vectors, and elements can be found, for example, in: Ausubel et al. (eds.), Current Protocols in Molecular Biology (1995) (John Wiley & Sons); Sambrook, Fritsch, & Maniatis (eds.), Molecular Cloning (1989) (Cold Spring Harbor Laboratory Press, NY); Berger & Kimmel, Methods in Enzymology 152: Guide to Molecular Cloning Techniques (1987) (Academic Press); Bukhari et al. (eds.), DNA Insertion Elements, Plasmids and Episomes (1977) (Cold Spring Harbor Laboratory Press, NY); Miller, J. H. Experiments in molecular genetics (1972) (Cold spring Harbor Laboratory Press, NY)

The embodiments described below are selected to illustrate the invention and are not limiting the invention in any way.

Media

The Luria Broth (LB) medium was made using LB Broth Powder, Millers (Fisher Scientific) and LB agar plates were made using LB Agar Powder, Millers (Fisher Scientific). When appropriated ampicillin ((100 μg/mL) or any appropriated antibiotic), and/or chloramphenicol (20 μg/mL) was added.

Basal Minimal medium had the following composition: NaOH (1 g/L), KOH (2.5 g/L), KH₂PO₄ (7 g/L), NH₄H₂PO₄ (7 g/L), Citric acid (0.5 g/l), Trace mineral solution (5 mL/L). The trace mineral stock solution contained: ZnSO₄*7H₂O 0.82 g/L, Citric acid 20 g/L, MnSO₄*H₂O 0.98 g/L, FeSO₄*7H₂O 3.925 g/L, CuSO₄*5H₂O 0.2 g/L. The pH of the Basal Minimal Medium was adjusted to 7.0 with 5 N NaOH and autoclaved. Before inoculation, the Basal Minimal medium was supplied with 1 mM MgSO₄, 4 μg/mL thiamine, 0.5% of a given carbon source (glucose or glycerol (Carbosynth)). Thiamine, and antibiotics, were sterilized by filtration. All percentage concentrations for glycerol are expressed as v/v and for glucose as w/v.

M9 plates containing 2-deoxy-galactose had the following composition: 15 g/L agar (Fisher Scientific), 2.26 g/L 5× M9 Minimal Salt (Sigma-Aldrich), 2 mM MgSO₄, 4 μg/mL thiamine, 0.2% glycerol, and 0.2% 2-deoxy-D-galactose (Carbosynth).

MacConkey indicator plates had the following composition: 40 g/L MacConkey agar Base (BD Difco™) and a carbon source at a final concentration of 1%.

Cultivation

Unless otherwise noted, E. coli strains were propagated in Luria-Bertani (LB) medium containing 0.2% glucose at 37° C. with agitation. Agar plates were incubated at 37° C. overnight.

Chemical Competent Cells and Transformations

E. coli was inoculated from LB plates in 5 mL LB containing 0.2% glucose at 37° C. with shaking until OD600˜0.4. 2 mL culture was harvested by centrifugation for 25 seconds at 13.000 g. The supernatant was removed and the cell pellet resuspended in 600 μL cold TB solutions (10 mM PIPES, 15 mM CaCl₂, 250 mM KCl). The cells were incubated on ice for 20 minutes followed by pelleting for 15 seconds at 13.000 g. The supernatant was removed and the cell pellet resuspended in 100 μL cold TB solution. Transformation of plasmids were done using 100 μL competent cells and 1 to 10 ng plasmid DNA. Cells and DNA were incubated on ice for 20 minutes before heat shocking at 42° C. for 45 seconds. After 2 min incubation on ice 400 μL SOC (20 g/L tryptone, 5 g/L Yeast extract, 0.5 g/L NaCl, 0.186 g/L KCl, 10 mM MgCl₂, 10 mM MgSO₄ and 20 mM glucose) was added and the cell culture was incubated at 37° C. with shaking for 1 hour before plating on selective plates.

Plasmids were transformed into TOP10 chemical competent cells at conditions recommended by the supplier (ThermoFisher Scientific).

DNA Techniques

Plasmid DNA from E. coli was isolated using the QIAprep Spin Miniprep kit (Qiagen). Chromosomal DNA from E. coli was isolated using the QIAmp DNA Mini Kit (Qiagen). PCR products were purified using the QIAquick PCR Purification Kit (Qiagen). DreamTaq PCR Master Mix (Thermofisher), Phusion U hot start PCR master mix (Thermofisher), USER Enzym (New England Biolab) were used as recommended by the supplier. Primers were supplied by Eurofins Genomics, Germany. PCR fragments and plasmids were sequenced by Eurofins Genomics. Colony PCR was done using DreamTaq PCR Master Mix in a T100 ™ Thermal Cycler (Bio-Rad).

TABLE 2
Oligos used for amplification of plasmid backbones, promoter elements, and genes of
interest (colonic acid genes, futA_mut4, futC, setA, marc, nec and vag)

| | SEQ | | |
|---|---|---|---|
| Name | ID NO | Oligonucleotide Sequence 5′-3′ | Description |
| O48 | 5 | CCCAGCGAGACCTGACCGCAGAAC | galK.for |
| O49 | 6 | CCCCAGTCCATCAGCGTGACTACC | galK.rev |
| O40 | 7 | ATTAACCCUCCAGGCATCAAATAAAACGAAAGGC | Backbone.for |
| O79 | 8 | ATTTGCGCAUCACCAATCAAATTCACGCGGCC | Backbone.rev |
| OL-0550 | 9 | TCCCCGCGCGTTGGCCGATTCATTAATGCAGCTGG | wcaJ::PglpF.for |
| | | CACGACAGGTTTCCCGACTGGAAAGCGGGCAGTGA | |
| | | GCGCAATGCGCAAATGCGGCACGCCTTGCAGATTA | |
| | | CG | |
| OL-0511 | 10 | TTTTTCCAGCAGAAACTCTGCCAGGTAAGAACCGTC | wcaJ::PglpF.rev |
| | | TTGTCCGGTTACACCGGTGATGAGAGCGACTTTTGA | |
| | | CATAGCTGTTTCCTCCTTGGTTAATGTTTGTTGTATG | |
| | | CG | |
| O261 | 11 | ATGCGCAAAUGCGGCACGCCTTGCAGATTACG | PglpF.for |
| O262 | 12 | AGCTGTTUCCTCCTTGGTTAATGTTTGTTGTATGCG | PglpF.rev |
| O364 | 13 | ATGCGCAAAUTGCGTCGCCATTCTGTCGCAACACG | PmglB_70UTR_SD4. |
| | | CC | for |
| O459 | 14 | AGCTGTTUCCTAGTTGGTTAATGTTTGTTGTATGCG | PmglB_70UTR_SD4. |
| | | | rev |
| O342 | 15 | AAACAGCUATGTCAAAAGTCGCTCTCATCACCGG | CA.for |
| O126 | 16 | AGGGTTAAUTGCGCGTTACTCGTTCAGCAACGTCAG | CA.rev |
| | | C | |
| O123 | 17 | AAACAGCUATGGCGTTCAAAGTGGTCCAAATC | futC.for |
| O124 | 18 | AGGGTTAAUTGCGCGTTAGCCCAGCGCGTTATATTT | futC.rev |
| | | CTG | |
| KABY528 | 19 | AAACAGCUATGTTCCAACCGCTGCTGGACG | futA_mut4.for |
| KABY568 | 20 | AGGGTTAAUTTACAGACCCAGTTTTTTGACCAGTTTA | futA_mut4.rev |
| | | CG | |
| O449 | 21 | AAACAGCUATGATCTGGATAATGACGAT | setA.for |
| O450 | 22 | AGGGTTAAUTCAAACGTCTTTAACCTTTGCGG | setA.rev |
| O737 | 23 | AAACAGCUATGCAGCGTCTGAGCCGTCTGAG | marc.for |
| O738 | 24 | AGGGTTAAUTTAAACTTCACGCACTTTCGCGC | marc.rev |
| O741 | 25 | AAACAGCUATGCAGAGCTTCACCCCGCC | nec.for |
| O742 | 26 | AGGGTTAAUTTACGCCTGCTCTTTAACACGCAGC | nec.rev |
| KABY745 | 27 | AAACAGCUATGAAGAGCCTGCTGACCCGTAAAC | vag.for |
| KABY746 | 28 | AGGGTTAAUTTAAACGTTTTTCACACGCGCG | vag.rev |
| KABY733 | 48 | AAACAGCUATGAAGAGCGCGCTGACCTTCAG | fred.for |
| KABY734 | 49 | AGGGTTAAUTTACGCTTCACGCACACGCG | fred.rev |
| KABY729 | 50 | AAACAGCUATGAGCAGCCGTCGTCTGAGC | bad.for |
| KABY730 | 51 | AGGGTTAAUTTACACGTTTTTAACACGGGTCATCAG | bad.rev |
| KABY721 | 52 | AAACAGCUATGAAGAGCGCGCTGACCTTTAGC | yberC.for |
| KABY722 | 53 | AGGGTTAAUTTACGCCTCACGCACACGCG | yberC.rev |

TABLE 3
The heterologous proteins expressed in the HMO-producing cells

| | | Protein GenBank | SEQ | |
|---|---|---|---|---|
| Gene | Origin of Genes | Accession Number | ID NO | Protein Function |
| futC* | Helicobacter pylori 26695 | WP_080473865.1 | 37 | alpha-1,2-fucosyl- |
| | | | | transferase |
| futA** | Helicobacter pylori 26695 | WP_000487428.1 | 38 | alpha-1,3-fucosyl- |
| | | | | transferase |
| fucT | Helicobacter pylori NCTC 11639 | AAB81031.1 | 40 | alpha-1,3-fucosyl- |
| | | | | transferase |
| moumou | Acanthamoeba polyphaga | YP_007354660 | 54 | alpha-1,3-fucosyl- |
| | moumouvirus | | | transferase |
| setA | Escherichia coli | YP_025293 | 41 | Sugar efflux |
| | | | | transporter |
| marc | Serratia marcescens | WP_060448169.1 | 1 | MFS transporter |
| nec | Rosenbergiella nectarea | WP_092672081.1 | 2 | MFS transporter |
| vag | Pantoea vagans | WP_048785139.1 | 3 | MFS transporter |
| Fred | Yersinia frederiksenii | WP_087817556.1 | 42 | MFS transporter |
| Bad | Rouxiella badensis | WP_017489914.1 | 43 | MFS transporter |
| yberC | Yersinia bercovieri | EEQ08298.1 | 44 | MFS transporter |

*FutC used herein has two additional amino acids (LG) at C-terminus
**futA mut4 has 6 amino acid modification: S46F A128N H129E Y132I D148G Y221C (SEQ ID NO: 39).

Alternative alpha 1,2-fucosyl transferases are wbgL from E. coli 0126 (NCBI accession nr ADN43847, disclosed in WO 2016/120448, hereby incorporated by reference) or fucT2 from Helicobacter pylori (NCBI ref AAC99764 hereby incorporated by reference).

TABLE 4
The synthetic DNA utilized in this the HMO-producing cells

| Sequence | SEQ | | |
|---|---|---|---|
| name | ID NO | Description | Reference |
| PmglB_70 | 4 | Promoter: 203-nucleotide DNA expression element | WO2020255054 |
| UTR_SD4 | | | |
| PglpF | 29 | Promoter: 300-nucleotide DNA expression element | WO2019123324 |
| CA | 30 | CA operon: 6.706-nucleotide fragment containing genes | WO2019123324 |
| | | gmd-wcaG-wcaH-wcal-manC-manB | |
| futC | 31 | alpha-1,2-fucosyl-transferase encoding sequence: | WO2019123324 |
| | | 909-nucleotide fragment containing gene futC | |
| futA_mut4 | 32 | alpha-1,3-fucosyl-transferase encoding sequence: | |
| | | 1.278-nucleotide fragment containing gene futA_mut4 | |
| setA | 33 | Sugar efflux transporter encoding sequence | NCBI: CP032679 |
| | | 1.179-nucleotide fragment containing gene setA | position 77620- |
| | | | 78798 |
| marc | 34 | MFS transporter encoding sequence: | WO2021148614 |
| | | 1.197 nucleotide fragment containing gene marc, | |
| nec | 35 | MFS transporter encoding sequence: | WO2021148615 |
| | | 1.185 nucleotide fragment containing gene nec, | |
| vag | 36 | MFS transporter encoding sequence: | WO2021148611 |
| | | 1.179 nucleotide fragment containing gene vag, | |
| Fred | 45 | MFS transporter encoding sequence: | WO2021148620 |
| | | 1.182 nucleotide fragment containing gene fred | |
| Bad | 46 | MFS transporter encoding sequence: | WO2021148618 |
| | | 1.164 nucleotide fragment containing gene bad | |
| yberC | 47 | MFS transporter encoding sequence: | WO2021148610 |
| | | 1.185 nucleotide fragment containing gene yberc | |

Construction of Plasmids

Plasmid backbones containing two I-Scel endonuclease sites, separated by two DNA fragments appropriated for homologous recombination into the E. coli genome and a T1 transcriptional terminator sequence were synthesized. For example, in one plasmid backbone the gal operon (required for homologous recombination in galK), and a T1 transcriptional terminator sequence (pUC57::gal) was synthesized (GeneScript). The DNA sequences used for homologous recombination in the gal operon covered base pairs 3.628.621-3.628.720 and 3.627.572-3.627.671 in sequence Escherichia coli K-12 MG155 complete genome GenBank: ID: CP014225.1. Insertion by homologous recombination would result in a deletion of 949 base pairs of galK and a galK-phenotype. In similar ways, backbones based on pUC57 (GeneScript) or an any other appropriated vector containing two I-Scel endonuclease sites, separated by two DNA fragments appropriated for homologous recombination into the E. coli genome and a T1 transcriptional terminator sequence could be synthesized. Standard techniques well-known in the field of molecular biology were used for designing of primers and amplification of specific DNA sequences of the Escherichia coli K-12 DH1 chromosomal DNA.

Chromosomal DNA obtained from E. coli K-12 DH1 was used to amplify a 300 bp DNA fragment containing the promoter PglpF using oligos 0261 and 0262 (Table 2) (described in WO2019123324).

A synthetic promoter element was constructed by fusion of the mglB promoter to the 70UTR_SD4 sequence of PglpF_SD4 resulted in a 203 bp promoter element, PmglB_70UTR_SD4 (Table 3, described in PCT/IB2020/055773). This promoter element was amplified using oligos 0364 and 0459 (Table 2).

Chromosomal DNA obtained from E. coli K-12 DH1 was used to amplify a 6.706 bp DNA fragment containing the colonic acid genes gmd-wcaG-wcaH-wcaI-manC-manB (Table 3) using oligos 0342 and 0126 (Table 2).

A 909 bp DNA fragment containing a codon optimized version of the futC gene originating from Helicobacter pylori 26695 was synthesised by GeneScript (Table 4). The futC gene was amplified by PCR using oligos 0123 and 0124 (Table 2).

A 1.278 bp DNA fragment containing a codon optimised version of the futA gene including eight modified base pairs was synthesised by GeneScript (Table 4). The futA_mut4 was amplified by PCR using oligos KABY528 and KABY568 (Table 2). The futA gene originates from Helicobacter pylori 26695.

A 1.179 bp DNA fragment containing setA originating from Escherichia coli K-12 DH1 (Table 4) was amplified by PCR using chromosomal DNA from Escherichia coli K-12 DH1 and oligos 0499 and 0450 (Table 2).

A 1.197 bp DNA fragment containing a codon optimized version of the marc gene originating from Serratia marcescens was synthesized by GeneScript (Table 4). The marc gene was amplified by PCR using oligos 0737 and 0738 (Table 2).

A 1.185 bp DNA fragment containing a codon optimized version of the nec gene originating from Rosenbergiella nectarea was synthesized by GeneScript (Table 4). The nec gene was amplified by PCR using oligos 0741 and 0742 (Table 2).

A 1.179 bp DNA fragment containing a codon optimized version of the vag gene originating from Pantoea vagans was synthesized by GeneScript (Table 4). The vag gene was amplified by PCR using oligos KABY745 and KABY746 (Table 2).

A 1.182 bp DNA fragment containing a codon optimized version of the fred gene, originating from Yersinia frederiksenii was synthesized by Genescript (Table 4). The fred gene was amplified using oligos KABY733 and KABY734 (Table 2).

A 1.182 bp DNA fragment containing a codon optimized version of the bad gene, originating from Rouxiella badensis was synthesized by Genescript (Table 4). The bad gene was amplified using oligos KABY729 and KABY730 (Table 2).

A 1.185 bp DNA fragment containing a codon optimized version of the yberC gene originating from Yersinia bercovieri was synthesized by GeneScript (Table 4). The yberC gene was amplified by PCR using oligos KABY721 and KABY722 (Table 2).

All PCR fragments (plasmid backbones, promoter elements and genes of interest were purified, and plasmid backbones, promoter elements, and genes of interest were assembled. The plasmids were cloned by standard USER cloning. Cloning in any appropriated plasmid could be done using any standard DNA cloning techniques. The plasmids were transformed into TOP10 cells and selected on LB plates containing 100 μg/mL ampicillin (or any appropriated antibiotic) and 0.2% glucose. The constructed plasmids were purified and the promoter sequence and the 5′end of the gene of interest was verified by DNA sequencing (MWG Eurofins Genomics). In this way, a genetic cassette containing any promoter of interest fused to any gene of interest was constructed and used for chromosomal integration by homologous recombineering.

Construction of Strains

The bacterial strain used, MDO, was constructed from Escherichia coli K-12 DH1. The E. coli K-12 DH1 genotype is: F⁻, λ⁻, gyrA96, recA1, relA1, endA1, thi-1, hsdR17, supE44. In addition to the E. coli K-12 DH1 genotype MDO has the following modifications: lacZ: deletion of 1.5 kbp, lacA: deletion of 0.5 kbp, nanKETA: deletion of 3.3 kbp, melA: deletion of 0.9 kbp, wcaJ: deletion of 0.5 kbp, mdoH: deletion of 0.5 kbp, and insertion of Plac promoter upstream of the gmd gene. Below is a description of the strain construction used in the present Examples. A summary of the strains can be found in table 5.

The plasmids containing the expression cassettes, PglpF-gmd-wcaG-wcaH-wcaI-manC-manB, PglpF-futC, PglpF-futA_mut4, Pmg18_70UTR_SD4-futC, PglpF-setA, PglpF-marc, PglpF-nec, or PglpF-vag were integrated into the chromosomal DNA by homologues recombineering as described in WO2019123324. Briefly, for integration in the chromosomal DNA the helper plasmid, pACBSR, and the donor plasmid containing the expression cassettes (as described above) were co-transformed into MDO and selected on LB plates containing 0.2% glucose, ampicillin (100 μg/ml) or kanamycin (50 mg/mL) and chloramphenicol (20 pg/ml). A single colony was inoculated in 1 ml LB containing chloramphenicol (20 pg/ml) and 10 μ l of 20% L-arabinose and incubated at 37° C. with shaking for 7-8 hours. Selection for insertion in the galK loci was done by plating on M9-DOG plates and incubated at 37° C. for 48 hours. Single colonies formed on MM-DOG plates were re-streaked on LB plates containing 0.2% glucose and incubated for 24 hours at 37° C. Colonies that appeared white on MacConkey-galactose agar plates and were sensitive for both ampicillin and chloramphenicol were expected to have lost the donor and the helper plasmid, and contain an insertion in the galK loci. Insertions in the galK site was identified by colony PCR using primers 048 and 049 located outside the galK loci. Chromosomal DNA was purified, the galK locus was amplified using primers 048 and 049 and the inserted DNA was verified by sequencing (Eurofins Genomics, Germany). A number of genetic cassettes were integrated into several specific loci in the chromosomal DNA using homologous DNA located upstream and downstream of the integration site of interest.

Strain 1 was constructed by inserting one genetic expression cassette containing PglpF fused to the colonic acid operon gmd-wcaG-wcaH-wcaI-manC-manB and inserting two genetic expression cassettes containing PglpF fused to futC into the chromosomal DNA of strain MDO. The lacI gene was replacement with a marker gene by homologous recombineering. The marker gene in lacI was removed again by homologous recombination resulting in scar-less removal of the lacI gene.

Strain 2 was constructed by replacing Plac located upstream of gmd with PglpF. First a marker gene replaced the Plac element by homologous recombineering and secondly the marker gene was replaced by PglpF by homologous recombineering using a dsDNA fragment constructed by PCR using oligos OL-0550 and OL-0511 on a DNA fragment containing PglpF. Furthermore, three genetic expression cassettes containing PglpF fused to futA_mut4, and one genetic expression cassette containing PglpF fused to marc were inserted at specific loci in the chromosomal DNA of strain MDO. The lacI gene was replacement with a marker gene by homologous recombineering. The marker gene in lacI was removed again by homologous recombination resulting in scar-less removal of the lacI gene.

Strain 3 was constructed as strain 2 except that Pmg18_70UTR_SD4 fused to futC was inserted into the chromosomal DNA of strain MDO instead of PglpF-marc.

Strain 4 was constructed by inserting one genetic expression cassette containing PglpF fused to setA into the chromosome of strain 3.

Strain 5 was constructed by inserting one genetic expression cassette containing PglpF fused to marc into the chromosome of strain 3.

Strain 6 was constructed by inserting one genetic expression cassette containing PglpF fused to nec into the chromosome of strain 3.

Strain 7 was constructed by inserting one genetic expression cassette containing PglpF fused to vag into the chromosome of strain 3.

Strain 8 was constructed by inserting one genetic expression cassette containing PglpF fused to futC into the chromosome of strain 2.

Strain 9 was constructed by inserting one genetic expression cassette containing PglpF fused to fred into the chromosome of strain 3.

Strain 10 was constructed by inserting one genetic expression cassette containing PglpF fused to bad into the chromosome of strain 3.

Strain 11 was constructed by inserting one genetic expression cassette containing PglpF fused to YberC into the chromosome of strain 3.

Strain 12 was constructed by transformation of a kanamycin resistant pTOPO plasmid construct comprising the 1,3-fucosyltransferase, futA, under control of the PglpF promoter and a transcriptional terminator (pl-futA-mut4), into strain 1A (2′-FL strain with the nec transporter).

Strain 13 was constructed by transformation of a kanamycin resistant pTOPO plasmid construct comprising the 1,3-fucosyltransferase, fucT, under control of the PglpF promoter and a transcriptional terminator (pl-fucT) into strain 1A (2′-FL strain with the nec transporter).

Strain 14 was constructed by transformation of a kanamycin resistant pTOPO plasmid construct comprising the 1,3-fucosyltransferase, moumou (table 1), under control of the PglpF promoter and a transcriptional terminator (pl-moumou), into strain 1A (2′-FL strain with the nec transporter).

Strain 15 was constructed by transformation of a kanamycin resistant pTOPO plasmid construct comprising the 1,3-fucosyltransferase, fucT, under control of the PglpF promoter and a transcriptional terminator (pl-fucT), into strain 1B (2′-FL strain with the marc transporter).

TABLE 5
Strain constructions

| Strain IDs | Product | Relevant Genotype |
|---|---|---|
| DH1 | — | F⁻ λ⁻ endA1 recA1 relA1 gyrA96 thi-1 glnV44 hsdR17(r_{K}⁻ m_{K}⁻) |
| MDO | — | E coli DH1 ΔlacZ ΔlacA ΔnanKETA ΔmelA ΔwcaJ ΔmdoH |
| Strain 1 | 2′FL | MDO PglpF-gmd-wcaG-wcaH-wcal-manC-manB ΔlacI 2xPglpF-futC |
| Strain 1A | | MDO PglpF-gmd-wcaG-wcaH-wcal-manC-manB ΔlacI 2xPglpF-futC PglpF- |
| | | nec |
| Strain 1B | | MDO PglpF-gmd-wcaG-wcaH-wcal-manC-manB ΔlacI 2xPglpF-futC PglpF- |
| | | marc |
| Strain 2 | 3FL | MDO wcaF::PglpF ΔlacI 3xPglpF-futA_mut4 PglpF-marc |
| Strain 3 | DFL | MDO wcaF::PglpF ΔlacI 3xPglpF-futA_mut4 PmglB_70UTR_SD4-futC |
| Strain 4 | | MDO wcaF::PglpF ΔlacI 3xPglpF-futA_mut4 PmglB_70UTR-futC_SD4 |
| | | PglpF-setA |
| Strain 5 | | MDO wcaF::PglpF ΔlacI 3xPglpF-futA_mut4 PmglB_70UTR-futC_SD4 |
| | | PglpF-marc |
| Strain 6 | | MDO wcaF::PglpF ΔlacI 3xPglpF-futA_mut4 PmglB_70UTR-futC_SD4 |
| | | PglpF-nec |
| Strain 7 | | MDO wcaF::PglpF ΔlacI 3xPglpF-futA_mut4 PmglB_70UTR-futC_SD4 |
| | | PglpF-vag |
| Strain 8 | | MDO wcaF::PglpF ΔlacI 3xPglpF-futA_mut4 PglpF-marc PglpF-futC |
| Strain 9 | | MDO wcaF::PglpF ΔlacI 3xPglpF-futA_mut4 PmglB_70UTR-futC_SD4 |
| | | PglpF-fred |
| Strain 10 | | MDO wcaF::PglpF ΔlacI 3xPglpF-futA_mut4 PmglB_70UTR-futC_SD4 |
| | | PglpF-bad |
| Strain 11 | | MDO wcaF::PglpF ΔlacI 3xPglpF-futA_mut4 PmglB_70UTR-futC_SD4 |
| | | PglpF-yberC |
| Strain 12 | | MDO PglpF-gmd-wcaG-wcaH-wcal-manC-manB ΔlacI 2xPglpF-futC PglpF- |
| | | nec containing plasmid pl-futA_mut4 |
| Strain 13 | | MDO PglpF-gmd-wcaG-wcaH-wcal-manC-manB ΔlacI 2xPglpF-futC PglpF- |
| | | nec containing plasmid pl-fucT |
| Strain 14 | | MDO PglpF-gmd-wcaG-wcaH-wcal-manC-manB ΔlacI 2xPglpF-futC PglpF- |
| | | nec containing plasmid pl-moumou |
| Strain 15 | | MDO PglpF-gmd-wcaG-wcaH-wcal-manC-manB ΔlacI 2xPglpF-futC PglpF- |
| | | marc containing plasmid pl-fucT |

Deep Well Assay (DWA)

DWA was performed as originally described to Lv et al (Bioprocess Biosyst Eng (2016) 39:1737-1747) and optimized for the purposes of the current invention.

More specifically, the strains disclosed in the examples were screened in 24 deep well plates using a 4-day protocol. During the first 24 hours, cells were grown to high densities while in the next 48 hours cells were transferred to a medium that allowed induction of gene expression and product formation. Specifically, during day 1 fresh inoculums were prepared using a basal minimal medium supplemented with magnesium sulphate, thiamine and glucose. After 24 hours of incubation of the prepared cultures at 34° C. with a 700 rpm shaking, cells were transferred to a new basal minimal medium (2 ml) supplemented with magnesium sulphate and thiamine to which an initial bolus of 20% glucose solution (1 μl) and 10% lactose solution (0.1 ml) were added, then 50% sucrose solution as carbon source was provided to the cells accompanied by the addition of sucrose hydrolase (invertase, 4 μl of a 0.1 g/L solution) so that glucose was provided at a slow rate for growth by cleavage of sucrose by the invertase. After inoculation of the new medium, cells were shaken at 700 rpm at 28° C. for 48 hours. After denaturation and subsequent centrifugation, the supernatants were analysed by HPLC.

Fermentation

Fermentations were carried out in 200 mL DasBox bioreactors (Eppendorf, Germany) or 2 L Biostat B bioreactors (Sartorius, Germany). Starting volumes, respectively, were 100 mL or 1 L. The medium was a defined minimal culture medium, consisting of 25 g/kg carbon source (glucose), MgSO₄×7H₂O, KOH, NaOH, NH₄H₂PO₄, KH₂PO₄, trace element solution, citric acid, antifoam and thiamine. The trace metal solution (TMS) contained Mn, Cu, Fe, Zn as sulfate salts and citric acid. Fermentations were started by inoculation with 2% (v/v) of pre-cultures grown in a defined minimal medium. After depletion of the carbon source contained in the batch medium, a sterile feed solution containing glucose, MgSO₄×7H₂O, TMS, H₃PO₄, antifoam and lactose was fed continuously in a glucose-limited manner, using a predetermined, linear profile. Lactose concentration in the feed solution was either 120 g/kg (process DFL1) or 60 g/kg (process DFL2), to obtain either high or low lactose concentrations during fermentation. Hence, low lactose condition was defined as having <5 g/I throughout most of the fermentation, while high lactose condition was defined as having 10-25 g/L throughout most of the fermentation. FIG. 4 depicts the resulting lactose concentrations measured in the fermentation broth using HPLC.

The pH throughout fermentation was controlled at 6.8 by titration with NH₄OH solution. Aeration was controlled at 1 vvm using air, and dissolved oxygen was kept above 20% of air saturation, controlled by the stirrer rate. At 3 h after glucose feed start, the fermentation temperature setpoint was lowered from 33° C. to 30° C. This temperature drop was conducted instantly or with a 1 hour linear ramp.

Throughout the fermentations, samples were taken in order to determine the concentration of 2′FL, 3FL, DFL, lactose and other minor by-products using HPLC. Total broth samples were diluted three-fold in deionized water and boiled for 20 minutes. This was followed by centrifugation at 17000 g for 3 minutes, where after the resulting supernatant was analysed by H PLC.

### Example 1. Engineering of Escherichia coli for HMO Production by Overexpressing α-1,2-fucosyltransferase and α-1,3-fucosyltransferase

Three strains producing either 2′FL, 3FL, or DFL, as the main product were constructed. Strain 1, a 2′FL producing strain, overexpress the colonic acid genes (gmd-wcaG-wcaH-wcaI-manC-manB) and the α-1,2-fucosyltransferase gene, futC. Strain 2, a 3FL producing strain overexpress the colonic acid genes (gmd-wcaG-wcaH-wcaI-manC-manB), the α-1,3-fucosyltransferase gene, futA_mut4, and the MFS gene, marc. Strain 3, a DFL producing strain, overexpress the colonic acid genes (gmd-wcaG-wcaH-wcaI-manC-manB), the α-1,3-fucosyltransferase gene futA_mut4, and the α-1,2-fucosyltransferase gene, futC.

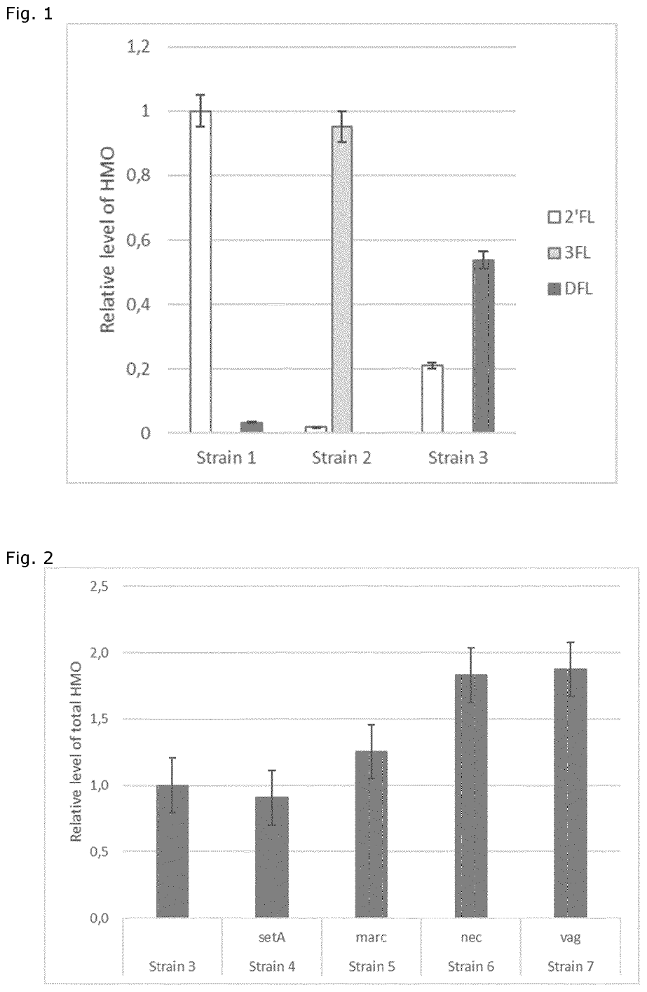

The strains were cultured using the deep well assay as described in the materials and method section and the contents of 2′FL, 3FL and DFL were measured using HPLC. The results are shown in FIG. 1.

Surprisingly, overexpressing the α-1,2-fucosyltransferase gene, futC, in a 3FL producing strain converts 3FL into DFL. More than 70% of the total HMO produced by strain 3 is DFL and the production of 3FL is almost eliminated.

As can be seen in FIG. 1, more than 70% of the total HMO produced by strain 3 is DFL and the production of 3FL is almost eliminated.

### Example 2. Engineering of Escherichia coli for DFL Production by Overexpression of a Heterologous MFS Protein

The main HMO produced by Strain 3 is DFL. Strain 3 overexpresses the colonic acid genes (gmd-wcaG-wcaH-wcaI-manC-manB), the α-1,2-fucosyltransferase gene, futC, and the α-1,3-fucosyltransferase, futA_mut4.

In the present example it was investigated whether overexpression of the homologous sugar efflux transporter protein, SetA (strain 4), or one of the three heterologous MFS transporter proteins, Marc (strain 5), Nec (strain 6), or Vag (strain 7), exporter proteins had an effect on total HMO expression and on the DFL/2′FL ratio compared to strain 3.

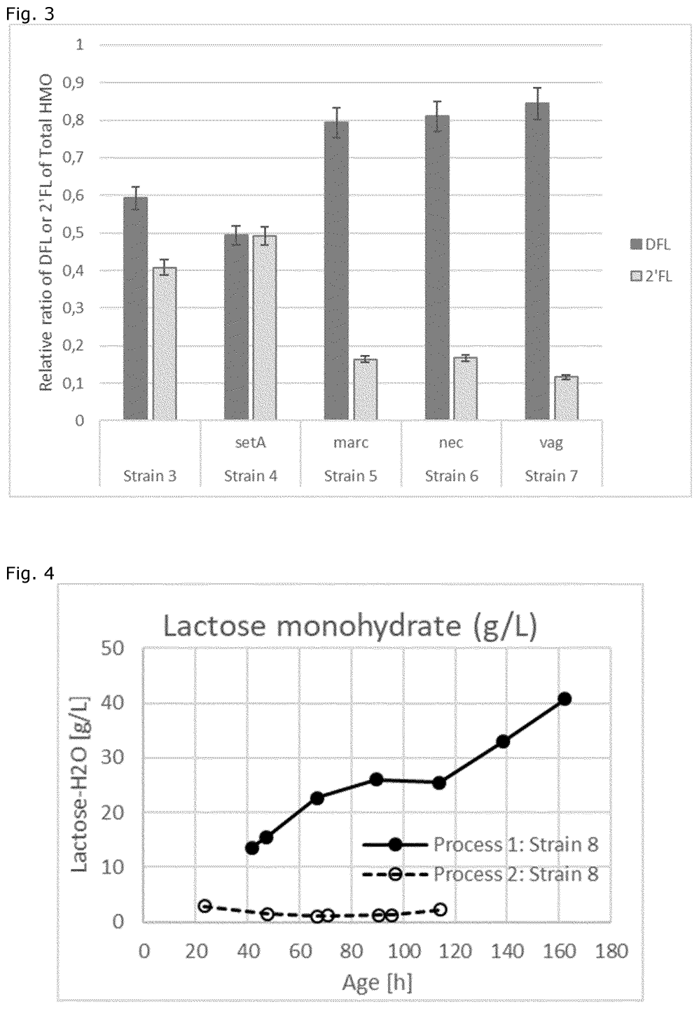

The strains were cultured using the deep well assay as described in the materials and method section and the contents of 2′FL, 3FL and DFL were measured using HPLC. The results are shown in FIGS. 2 and 3.

Overexpression of setA gene (Strain 4) did not increase the total amount of HMO produced (FIG. 2). Overexpression of the marc gene (Strain 5) increased the total amount of HMO produced by 25% (FIG. 2). Overexpression of the nec or vag gene, Strain 6 or 7, respectively, increased the total amount of HMO produced by 80% (FIG. 2). Furthermore, overexpression of marc, nec, or vag increased the ratio of DFL to the total amount of HMO by 25% (FIG. 3) and decreased the ratio of 2′FL compared to the total amount of HMO by more than 30% (FIG. 3). More than 70% of the produced HMOs in strains overexpressing the α-1,2-fucosyltransferase gene, futC, the α-1,3-fucosyltransferase, futA_mut4 combined with overexpression of either marc, nec, or vag, is DFL.

### Example 3. High Ratio of DFL:2′FL Obtained by Fermentation

Lactose is the substrate for the fucosylation performed by the alpha-1,2-fucosyl transferase and alpha-1,3-fucosyl transferase involved in DFL formation. In the present example it was investigated if the concentration of lactose in the feed during fermentation affected the DFL formation.

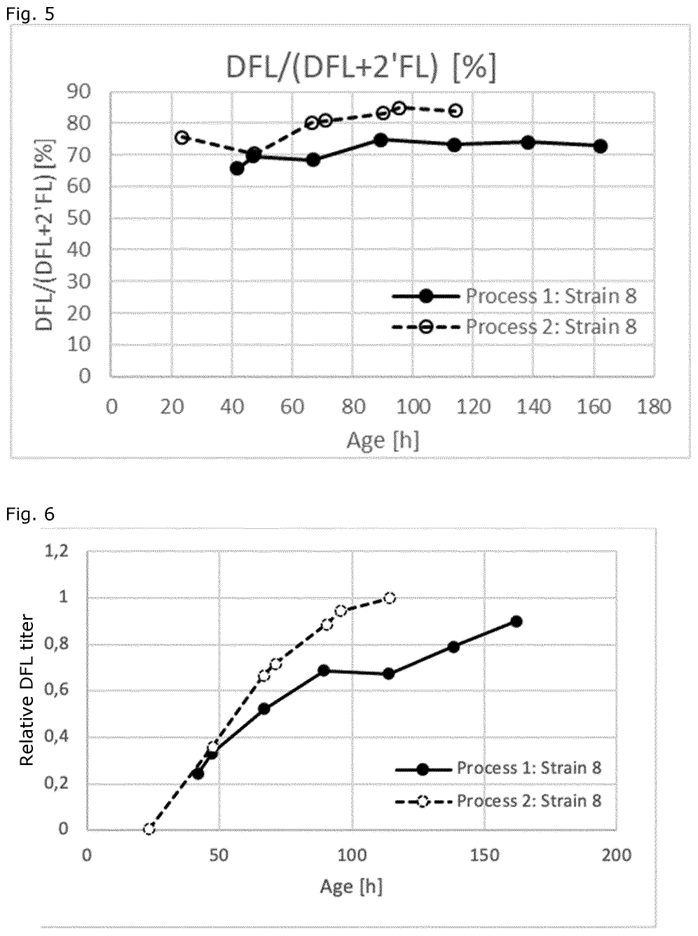

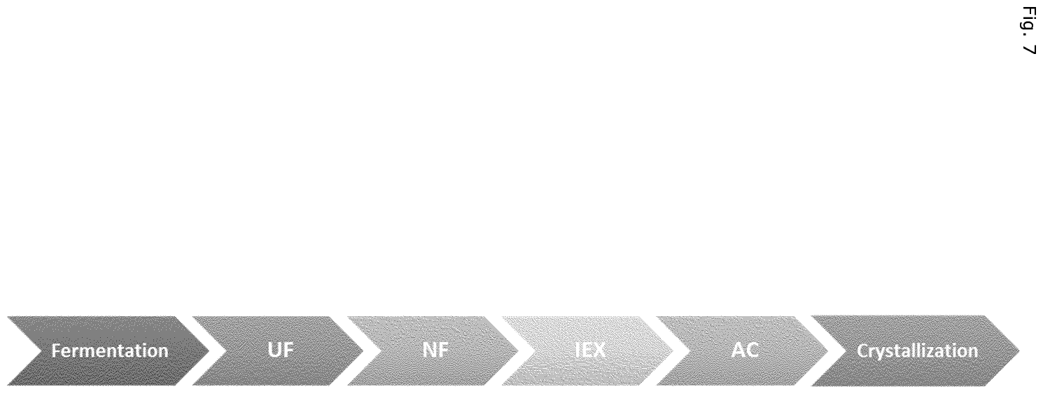

A DFL producing strain, strain 8, was capable of producing a mixture of 2′FL and DFL, where DFL is the predominant HMO, and 2′FL generally constitutes 30% or less of the total HMO, depending on the fermentation conditions, as described below. Surprisingly, almost no 3FL is detected in fermentations with these strains even though the alpha-1,3 fucosyltransferase gene futA_mut4 was expressed. Two fermentations with different supplies of lactose were run in parallel as described in the material and method section. The two fermentation processes were identical with regards to medium composition, glucose feed profile and fermentation process parameters such as temperature, pH and dissolved oxygen. The resulting lactose concentrations, as measured in the fermentation broth by HPLC, were above 15 g/L with process DFL1 and below 5 g/L with process DFL2 for most of the time during fermentation (FIG. 4). The low lactose process leads to the highest DFL/(2′FL+DFL) ratios of >80%, where this ratio stabilises towards the end of fermentation (FIG. 5). For the high lactose process (DFL1), the DFL/(2′FL+DFL) ratio is somewhat lower, but still >70%, which means that in all cases DFL is by far the most abundant HMO produced (FIG. 6). Surprisingly, 3FL is in all cases determined to be <1% of the total sum of HMO and therefore negligible for final product quality (table 6).

TABLE 6
HMO composition in total broth sample at
end-of-fermentation timepoint. HMO = sum of
2'FL and DFL, while 3FL is negligible at <1% in all samples.

| | | | DFL/ | 3FL/ | 2'FL/ |
|---|---|---|---|---|---|
| Fermentation | | Process ID | HMO | HMO | HMO |
| Batch IDGDF | Strain | (lactose high/low) | (%) | (%) | (%) |
| 19558 | Strain 8 | DFL1 (high) | 73.0 | <1 | 27.0 |
| 20090 | Strain 8 | DFL2 (low) | 83.8 | <1 | 16.2 |

### Example 4. Purification and Crystallization of DFL from Fermentation Broth

Following fermentation cells and proteins were removed by ultrafiltration and the obtained solution was concentrated by nanofiltration. The solution was eluted through a strong cation exchange resin (H⁺ form) and a weak anion exchange resin (free base form) to demineralize it. The solution was then treated with charcoal to decolorize it. Subsequently, the solution was concentrated at reduced pressure to the required concentration for the crystallization step. For crystallization of DFL ethanol (˜1.3 volumes) was added to the concentrated solution. The solution was seeded and stirred at room temperature for 18 hours. Subsequently, ethanol (˜1.3 volumes) was added continuously over 3 hours at room temperature. The crystals were filtered off and washed with ethanol (˜0.4 volumes). The crystals were dried on air until constant weight. DFL content (water free)>90% w/w %.

FIG. 7 shows the purification steps of the fermentation broth to obtain crystalline DFL.

Ultrafiltration (UF) is used to separate biomass from the broth, nanofiltration (NF) to concentrate the broth, ion absorbance step to remove salts and activated charcoal (AC) to remove color. Selective DFL crystallization as the final step provides DFL in very high purity.

### Example 5—Comparative Study of Different a Heterologous Transporter Proteins

In Example 2 the three MFS transporter proteins Marc, Nec, or Vag and the sugar efflux transporter protein, SetA were tested for their ability to increase DFL expression when inserted into strain 3. In the present example three additional MFS transporters Fred, Bad and YberC were overexpressed in the DFL producing strain (Strain 3) resulting in strain 9-11, respectively.

The strains were cultured using the deep well assay as described in the materials and method section and the contents of 2′FL, 3FL and DFL were measured using HPLC.

The percentage of DFL, 2FL and 3FL of the total amount of HMO produced is shown in table 7.

TABLE 7
Level of DFL, 2'FL or 3FL in % of total HMO produced
with different MFS transporters

| | | DFL | 2′FL | 3FL |
|---|---|---|---|---|
| | | % of total | % of total | % of total |
| Strain | Transporter used: | HMO | HMO | HMO |
| 1 | none | 59.6% | 39.3% | 1.1% |
| 4 | setA | 60.3% | 38.7% | 0.9% |
| 5 | marc | 67.3% | 27.3% | 5.4% |
| 6 | nec | 72.9% | 20.4% | 6.7% |
| 7 | vag | 69.9% | 19.7% | 10.5% |
| 9 | fred | 66.8% | 25.1% | 8.0% |
| 10 | bad | 71.3% | 24.6% | 4.1% |
| 11 | yberC | 60.5% | 34.9% | 4.5% |

As observed in example 2 overexpression of setA gene (Strain 4) did not increase the amount of DFL produced, which is also the case for the new exporter YberC (strain 11). As in Example 2, overexpression of marc, nec, or vag (strains 5-7) increased the ratio of DFL to the total amount of HMO by 7-12%. The same was observed for the new transporters fred and bad (strains 9 and 10). More than 65% of the produced HMOs in the strains with the marc, nec, vag fred or bad transporter proteins strains overexpressing the α-1,2-fucosyltransferase gene, futC, the α-1,3-fucosyltransferase, futA_mut4 is DFL.

### Example 6—Alternative α-1,3-Fucosyltransferase for the DFL Formation

The α-1,3-fucosyltransferase is responsible for the addition of fucosyl to the glucose moiety of the lactose substrate. In the following example the addition alternative α-1,3-fucosyltransferases in combination with the MFS transporter nec was tested.

Briefly the 2′-FL producing strain, strain 1 containing the FutC α-1,2-fucosyltransferase on the chromosome, was modified by overexpressing the nec MFS transporter protein generating strain 1A. To convert this 2′FL expressing strain to different a DFL expressing strains the cells were transfected with plasmids containing different α-1,3-fucosyltransferases.

The strains were cultured using the deep well assay as described in the materials and method section and the contents of 2′FL, 3FL and DFL were measured using HPLC. The results are shown in table 8.

TABLE 8
Level of DFL, 2'FL or 3FL in % of total HMO produced with
nec MFS transporter and different α-1,3-fucosyltransferase.

| | | | DFL | 2′FL | 3FL |
|---|---|---|---|---|---|
| | Transporter | α-1,3- | % of total | % of total | % of total |
| Strain | used: | fucosyltransferase | HMO | HMO | HMO |
| 1A | nec | none | 2.1 | 97.9 | 0.0 |
| 12 | nec | FutA_mut4 | 61.1 | 2.2 | 36.8 |
| 13 | nec | FucT | 64.2 | 3.4 | 32.4 |
| 14 | nec | moumou | 47.2 | 6.0 | 46.8 |

From this it can be seen that three different α-1,3-fucosyltransferase are capable of producing DFL as the most abundant HMO in the HMO mixture produced by the cell when combined with the nec MFS transporter. The moumou α-1,3-fucosyltransferase (strain 14) produces almost an equal amount of DFL and 3FL, the ratio may likely be changed towards DFL by adjusting the ratio between moumou α-1,3-fucosyltransferase and the α-1,2-fucosyltransferase FutC, since the moumou transferase is expressed from a high-expression plasmid and the FutC is expressed from 2 copies on the genome, so it would be expected that reducing the copy number of the moumou transferase would shift the HMO production towards more DFL and less 3FL. For the FutA_mut4 α-1,3-fucosyltransferase (strain 12) the high overexpression of FutA_mut4 from a high copy number plasmid in a strain comprising two copies of FutC results 61% DFL of total HMO. However, when compared to strain 6 in example 5 where only 3 copies of FutA_mut4 and one copy of futC was present in the strain, an increased production of 3FL and less DFLis produced by strain 12. This indicates that optimization of the fucosyltransferase ratios may be beneficial to increase the amount of DFL in the culture.

### Example 7—FucT α-1,3-fucosyltransferase in Combination with Marc MFS Transporter for the DFL Formation

In the following example the best performing α-1,3-fucosyltransferase from example 6, FucT, was tested in combination with the marc MFS transporter.

Briefly the 2′-FL producing strain, strain 1 containing the FutC α-1,2-fucosyltransferase on the chromosome, was modified by overexpressing the marc MFS transporter protein generating strain 1B. To convert this 2′FL expressing strain to a DFL expressing strain the strain was transfected with a plasmid containing the FucT α-1,3-fucosyltransferase.

The strains were cultured using the deep well assay as described in the materials and method section and the contents of 2′FL, 3FL and DFL were measured using HPLC. The results are shown in table 9.

TABLE 9
Level of DFL, 2'FL or 3FL in % of total HMO produced
using FucT α-1,3-fucosyltransferase

| | | | DFL | 2′FL | 3FL |
|---|---|---|---|---|---|
| | Transporter | α-1,3- | % of total | % of total | % of total |
| Strain | used: | fucosyltransferase | HMO | HMO | HMO |
| 1B | marc | none | 5.7 | 94.3 | 0.0 |
| 15 | marc | FucT | 75.9 | 12.1 | 12.0 |
| 13* | nec | FucT | 64.2 | 3.4 | 32.4 |

*Result from example 6

From these data it can be seen that the FucT α-1,3-fucosyltransferase is very efficient in producing DFL when combined with the α-1,2-fucosyltransferase FutC and the marc MFS transporter. DFL constitutes 76% of the HMO mix produced by the cell whereas 64% DFL was produced using FucT and the nec MFS transporter (data from table 8 included in table 9).

1. A genetically modified cell capable of producing one or more Human Milk Oligosaccharides (HMOs), wherein said cell comprises a heterologous, recombinant and/or synthetic nucleic acid encoding
a. an α-1,2-fucosyltransferase, and
b. an α-1,3-fucosyltransferase, and
c. a recombinant transporter protein selected from the major facilitator superfamily (MFS).

2. The genetically modified cell according to claim 1, wherein the genetically modified cell with the MFS transporter protein produces at least 5% w/w more DFL compared to the same cell without the MFS transporter protein.

3. The genetically modified cell according to claim 1, wherein the most abundant HMO produced by the genetically modified cell is difucosyllactose (DFL).

4. The genetically modified cell according to claim 1, wherein more than 60% w/w of the HMOs produced by the cell is difucosyllactose (DFL).

5. The genetically modified cell according to claim 1, wherein said heterologous, recombinant and/or synthetic nucleic acid encoding an α-1,2-fucosyltransferase is a futC gene or a wbgL A gene, or a functional homologue thereof.

6. The genetically modified cell according to claim 1, wherein said heterologous, recombinant and/or synthetic nucleic acid encoding an α-1,3-fucosyltransferase is a futA gene or a fucT gene or moumou gene, or a functional homologue thereof.

7. The genetically modified cell according to claim 1, wherein at the most 35% w/w of the total amount of the HMOs produced in the cell is 3-fucosyllactose (3FL), or 2′-fucosyllactose (2′FL).

8. The genetically modified cell according to claim 1, wherein the MFS transporter protein originates from a bacterium selected from the group consisting of Serratia marcescens, Rosenbergiella nectarea, Pantoea vagans, Yersinia frederiksenii and Rouxiella badensis.

9. The genetically modified cell according to claim 1, wherein the transporter protein is selected from the group consisting of SEQ ID NO: 1 (Marc), SEQ ID NO: 2 (Nec), SEQ ID NO: 3 (Vag), SEQ ID NO: 37 (fred) and SEQ ID NO: 38 (bad) or a functional homologue thereof which amino acid sequence is at least 80%, such as at least 85% or at least 90% identical to SEQ ID NO: 1 (Marc), SEQ ID NO: 2 (Nec), SEQ ID NO: 3 (Vag), SEQ ID NO: 42 (fred) or SEQ ID NO: 43 (bad).

10. The genetically modified cell according to claim 1, wherein the genetically modified cell is a microbial cell, such as Escherichia coli.

11. The genetically modified cell according to claim 1, wherein the cell further comprises a heterologous, recombinant and/or synthetic regulatory element selected from the group of a promoter nucleic sequences consisting of a Plac promoter, a PmglB promoter, a and a Pglp promoter, such as PglpF, or any variants thereof.

12. The genetically modified cell according to claim 11, wherein the regulatory element for the regulation of the expression of the α-1,2-fucosyltransferase comprises a promoter nucleic sequence which is PglpF or a variant thereof.

13. The genetically modified cell according to claim 11, wherein the regulatory element for the regulation of the expression of the α-1,3-fucosyltransferase comprises a promoter nucleic sequence which is PmglB or a variant thereof.

14. A method for the production of one or more HMOs, wherein the HMO produced is primarily difucosyllactose (DFL), the method comprising the steps of:
(i) providing a genetically modified cell according to claim 1
(ii) culturing the cell according to (i) in a suitable cell culture medium to produce said HMO; and
(iii) harvesting one or more HMOs produced in step (ii).

15. The method according to claim 14, wherein the method produces at least 5% w/w more DFL compared to the same method wherein the genetically modified cell differs from the cell in step (i) by not expressing the recombinant MFS transporter protein.

16. The method according to claim 14, wherein at the most 45%, such as at the most 30% w/w of the total amount of the HMOs produced in the cell is 3-fucosyllactose (3FL) and/or 2′-fucosyllactose (2′FL).

17. The method according to claim 14, wherein the culturing of the cell in step (ii) is conducted at low lactose conditions, such as conditions having less than 5 g lactose/1 culture medium.

18. Use of a genetically modified cell according to claim 1 for the production of one or more HMO, wherein the HMO produced is primarily difucosyllactose (DFL).

19. A 1,3-fuscosyl transferase with an amino acid sequence that is at least 90%, such as at least 95%, such as at least 98% identical to SEQ ID NO: 38 and which comprises or consists of the following substitutions S46F, A128N, H129E, Y1321, D148G and Y221C.

20. The 1,3-fuscosyl transferase according to claim 19, wherein the amino acid sequence comprises or consist of SEQ ID NO: 39.


SEQUENCE LISTING (54 sequences)

SEQ ID NO: 1 - protein, 396 aa, Serratia marcescens
MQRLSRLSLRINPIFAAFLLIAFLSGIAGALLTPTLSLFLTTEVKVRPLWVGLFYTANAVAGIVVSFLLAKRSDTRGDRRRLILLCCLMAVGNCLLFAFNRDYLTLITAGVLMSAVANTAMPQIFALAREYADSEAREVVMFSSVMRAQLSLAWVIGPPLSFALALNYGFTVMFLIAAVTFAVCVLLVGFMLPSVPRAAENEGLQGGVSAPIAPASAWRNRDVRLLFIASMLMWTCNTLYIIDMPLYITADLGLPEGLAGVLMGTAAGLEIPAMLLAGYYVKRFGKRNMMLLAVVAGVLFYLGLTVLESKPALIALQLLNAVFIGIVAGIGMLYFQDLMPGRPGAATTLFTNSISTGVILAGVLQGALVENLGHGSVYWMAALLALAALGMSAKVR

SEQ ID NO: 2 - protein, 394 aa, Rosenbergiella nectarea
MQSFTPPAPKGGNPVFMMFMLVTFFVSIAGALQAPTLSLYLSQELAAKPFMVGLFFTINAVTGIIISFILAKRSDRKGDRRRLLMFCCAMAIANALMFAFVRQYVVLITLGLILSALTSVVMPQLFALAREYADRTGREVVMFSSVMRTQMSLAWVIGPPISFALALNYGFITLYLVAAALFLLSLILIKTTLPSVPRLYPAEDLAKSAASGWKRTDVRFLFAASVLMWVCNLMYIIDMPLYISKSLGMPESFAGVLMGTAAGLEIPVMLLAGYLAKRVGKRPLVIVAAVCGLAFYPAMLVFHQQTGLLIIQLLNAVFIGIVAGLVMLWFQDLMPGKAGAATTLFTNSVSTGMIFAGLCQGLLSDLLGHQAIYVLATVLMVIALLLLLRVKEQA

SEQ ID NO: 3 - protein, 392 aa, Pantoea vagans
MKSLLTRKRRINPVFLAFMAASFMIGVAGALQAPTLSLFLTREVQARPLWVGLFFTVNAIAGIVVSMLVAKRSDSRGDRRTLILFCCAMAFCNALLFAFTRHYLTLITLGVLLSALASVSMPQIFALAREYADQSAREAVMFSSVMRAQLSLAWVIGPPLSFALALNFGFVTLFLVAAALFLVCILLIKFTLPSVPRAEPLMRSGGMPLSGWRDRDVRLLFIASVTMWTCNTMYIIDMPLYISVTLGLPEKLAGLLMGTAAGLEIPVMLLAGHYAKRVGKRNLMLIAVAAGVLFYAGLAMFASQTALMALQLFNAVFIGIIAGIGMLWFQDLMPGRPGAATTMFTNSISTGMILAGVIQGTLSERFGHIAVYWLALGLAVAAFAMSARVKNV

SEQ ID NO: 4 - DNA, 203 bp, Artificial Sequence
  PmglB_70UTR_SD4 203-nucleotide DNA expression element
tgcgtcgccattctgtcgcaacacgccagaatgcggcggcgatcactaactcaacaaatcaggcgatgtaaccgctttcaatctgtgagtgatttcacagtatcttaacaatgtgatagctatgattgcaccgtgcctacaagcatcgtggaggtccgtgactttcacgcatacaacaaacattaaccaactaggaaacagct

SEQ ID NO: 5 - DNA, 24 bp, Artificial Sequence
  O48_galK.for
cccagcgagacctgaccgcagaac

SEQ ID NO: 6 - DNA, 24 bp, Artificial Sequence
  O49_galK.rev
ccccagtccatcagcgtgactacc

SEQ ID NO: 7 - DNA, 34 bp, Artificial Sequence
  O40_backbone.for
  misc_RNA (9)..(9): /standard_name=“uracil”
attaacccuccaggcatcaaataaaacgaaaggc

SEQ ID NO: 8 - DNA, 32 bp, Artificial Sequence
  O79_Backbone.rev
  misc_RNA (10)..(10): /standard_name=“uracil”
atttgcgcaucaccaatcaaattcacgcggcc

SEQ ID NO: 9 - DNA, 107 bp, Artificial Sequence
  OL-0550_wcaJ::PglpF.for
tccccgcgcgttggccgattcattaatgcagctggcacgacaggtttcccgactggaaagcgggcagtgagcgcaatgcgcaaatgcggcacgccttgcagattacg

SEQ ID NO: 10 - DNA, 111 bp, Artificial Sequence
  OL-0511_wcaJ::PglpF.rev
tttttccagcagaaactctgccaggtaagaaccgtcttgtccggttacaccggtgatgagagcgacttttgacatagctgtttcctccttggttaatgtttgttgtatgcg

SEQ ID NO: 11 - DNA, 32 bp, Artificial Sequence
  O261_PglpF.for
  misc_RNA (10)..(10): /standard_name=“uracil”
atgcgcaaaugcggcacgccttgcagattacg

SEQ ID NO: 12 - DNA, 36 bp, Artificial Sequence
  O262_PglpF.rev
  misc_RNA (8)..(8): /standard_name=“uracil”
agctgttucctccttggttaatgtttgttgtatgcg

SEQ ID NO: 13 - DNA, 37 bp, Artificial Sequence
  O364_PmglB_70UTR_SD4.for
  misc_RNA (10)..(10): /standard_name=“uracil”
atgcgcaaautgcgtcgccattctgtcgcaacacgcc

SEQ ID NO: 14 - DNA, 36 bp, Artificial Sequence
  O459_PmglB_70UTR_SD4.rev
  misc_RNA (8)..(8): /standard_name=“uracil”
agctgttucctagttggttaatgtttgttgtatgcg

SEQ ID NO: 15 - DNA, 34 bp, Artificial Sequence
  O342_CA.for
  misc_RNA (8)..(8): /standard_name=“uracil”
aaacagcuatgtcaaaagtcgctctcatcaccgg

SEQ ID NO: 16 - DNA, 37 bp, Artificial Sequence
  O126_CA.rev
  misc_RNA (9)..(9): /standard_name=“uracil”
agggttaautgcgcgttactcgttcagcaacgtcagc

SEQ ID NO: 17 - DNA, 32 bp, Artificial Sequence
  O123_futC.for
  misc_RNA (8)..(8): /standard_name=“uracil”
aaacagcuatggcgttcaaagtggtccaaatc

SEQ ID NO: 18 - DNA, 39 bp, Artificial Sequence
  O124_futC.rev
  misc_RNA (9)..(9): /standard_name=“uracil”
agggttaautgcgcgttagcccagcgcgttatatttctg

SEQ ID NO: 19 - DNA, 30 bp, Artificial Sequence
  KABY528_futA_mut4.for
  misc_RNA (8)..(8): /standard_name=“uracil”
aaacagcuatgttccaaccgctgctggacg

SEQ ID NO: 20 - DNA, 39 bp, Artificial Sequence
  KABY568_futA_mut4.rev
  misc_RNA (9)..(9): /standard_name=“uracil”
agggttaauttacagacccagttttttgaccagtttacg

SEQ ID NO: 21 - DNA, 28 bp, Artificial Sequence
  O449_setA.for
  misc_RNA (8)..(8): /standard_name=“uracil”
aaacagcuatgatctggataatgacgat

SEQ ID NO: 22 - DNA, 32 bp, Artificial Sequence
  O450_setA.rev
  misc_RNA (9)..(9): /standard_name=“uracil”
agggttaautcaaacgtctttaacctttgcgg

SEQ ID NO: 23 - DNA, 31 bp, Artificial Sequence
  O737_marc.for
  misc_RNA (8)..(8): /standard_name=“uracil”
aaacagcuatgcagcgtctgagccgtctgag

SEQ ID NO: 24 - DNA, 32 bp, Artificial Sequence
  O738_marc.rev
  misc_RNA (9)..(9): /standard_name=“uracil”
agggttaauttaaacttcacgcactttcgcgc

SEQ ID NO: 25 - DNA, 28 bp, Artificial Sequence
  O741_nec.for
  misc_RNA (8)..(8): /standard_name=“uracil”
aaacagcuatgcagagcttcaccccgcc

SEQ ID NO: 26 - DNA, 34 bp, Artificial Sequence
  O742_nec.rev
  misc_RNA (9)..(9): /standard_name=“uracil”
agggttaauttacgcctgctctttaacacgcagc

SEQ ID NO: 27 - DNA, 33 bp, Artificial Sequence
  KABY745_vag.for
  misc_RNA (8)..(8): /standard_name=“uracil”
aaacagcuatgaagagcctgctgacccgtaaac

SEQ ID NO: 28 - DNA, 31 bp, Artificial Sequence
  KABY746_vag.rev
  misc_RNA (9)..(9): /standard_name=“uracil”
agggttaauttaaacgtttttcacacgcgcg

SEQ ID NO: 29 - DNA, 300 bp, Artificial Sequence
  300-nucleotide DNA expression element (PglpF)
gcggcacgccttgcagattacggtttgccacacttttcatccttctcctggtgacataatccacatcaatcgaaaatgttaataaatttgttgcgcgaatgatctaacaaacatgcatcatgtacaatcagatggaataaatggcgcgataacgctcattttatgacgaggcacacacattttaagttcgatatttctcgtttttgctcgttaacgataagtttacagcatgcctacaagcatcgtggaggtccgtgactttcacgcatacaacaaacattaaccaaggaggaaacagct

SEQ ID NO: 30 - DNA, 6706 bp, Escherichia coli
atgtcaaaagtcgctctcatcaccggtgtaaccggacaagacggttcttacctggcagagtttctgctggaaaaaggttacgaggtgcatggtattaagcgtcgcgcatcgtcattcaacaccgagcgcgtggatcacatttatcaggatccgcacacctgcaacccgaaattccatctgcattatggcgacctgagtgatacctctaacctgacgcgcattttgcgtgaagtacagccggatgaagtgtacaacctgggcgcaatgagccacgttgcggtctcttttgagtcaccagaatataccgctgacgtcgacgcgatgggtacgctgcgcctgctggaggcgatccgcttcctcggtctggaaaagaaaactcgtttctatcaggcttccacctctgaactgtatggtctggtgcaggaaattccgcagaaagagaccacgccgttctacccgcgatctccgtatgcggtcgccaaactgtacgcctactggatcaccgttaactaccgtgaatcctacggcatgtacgcctgtaacggaattctcttcaaccatgaatccccgcgccgcggcgaaaccttcgttacccgcaaaatcacccgcgcaatcgccaacatcgcccaggggctggagtcgtgcctgtacctcggcaatatggattccctgcgtgactggggccacgccaaagactacgtaaaaatgcagtggatgatgctgcagcaggaacagccggaagatttcgttatcgcgaccggcgttcagtactccgtgcgtcagttcgtggaaatggcggcagcacagctgggcatcaaactgcgctttgaaggcacgggcgttgaagagaagggcattgtggtttccgtcaccgggcatgacgcgccgggcgttaaaccgggtgatgtgattatcgctgttgacccgcgttacttccgtccggctgaagttgaaacgctgctcggcgacccgaccaaagcgcacgaaaaactgggctggaaaccggaaatcaccctcagagagatggtgtctgaaatggtggctaatgacctcgaagcggcgaaaaaacactctctgctgaaatctcacggctacgacgtggcgatcgcgctggagtcataagcatgagtaaacaacgagtttttattgctggtcatcgcgggatggtcggttccgccatcaggcggcagctcgaacagcgcggtgatgtggaactggtattacgcacccgcgacgagctgaacctgctggacagccgcgccgtgcatgatttctttgccagcgaacgtattgaccaggtctatctggcggcggcgaaagtgggcggcattgttgccaacaacacctatccggcggatttcatctaccagaacatgatgattgagagcaacatcattcacgccgcgcatcagaacgacgtgaacaaactgctgtttctcggatcgtcctgcatctacccgaaactggcaaaacagccgatggcagaaagcgagttgttgcagggcacgctggagccgactaacgagccttatgctattgccaaaatcgccgggatcaaactgtgcgaatcatacaaccgccagtacggacgcgattaccgctcagtcatgccgaccaacctgtacgggccacacgacaacttccacccgagtaattcgcatgtgatcccagcattgctgcgtcgcttccacgaggcgacggcacagaatgcgccggacgtggtggtatggggcagcggtacaccgatgcgcgaatttctgcacgtcgatgatatggcggcggcgagcattcatgtcatggagctggcgcatgaagtctggctggagaacacccagccgatgttgtcgcacattaacgtcggcacgggcgttgactgcactatccgcgagctggcgcaaaccatcgccaaagtggtgggttacaaaggccgggtggtttttgatgccagcaaaccggatggcacgccgcgcaaactgctggatgtgacgcgcctgcatcagcttggctggtatcacgaaatctcactggaagcggggcttgccagcacttaccagtggttccttgagaatcaagaccgctttcgggggtaatgatgtttttacgtcaggaagactttgccacggtagtgcgctccactccgcttgtctctctcgactttattgtcgagaacagtcgcggcgagtttctgcttggcaaaagaaccaaccgcccggcgcagggttactggtttgtgccgggagggcgcgtgcagaaagacgaaacgctggaagccgcatttgagcggctgacgatggcggaactggggctgcgtttgccgataacagcaggccagttttacggtgtctggcagcacttttatgacgataacttctctggcacggatttcaccactcactatgtggtgctcggttttcgcttcagagtatcggaagaagagctgttactgccggatgagcagcatgacgattaccgctggctgacgtcggacgcgctgctcgccagtgataatgttcatgctaacagccgcgcctattttctcgctgagaagcgtaccggagtacccggattatgaaaatactggtctacggcattaactactcgccggagttaaccggcatcggcaaatacaccggcgagatggtggaatggctggcggcacaaggtcatgaggtgcgggtcattaccgcaccgccttactacccgcaatggcaggtgggcgagaactattccgcctggcgctacaaacgagaagagggggccgccacggtgtggcgctgcccgctgtatgtgccaaaacagccgagcaccctgaaacgcctgttgcatctgggcagttttgccgtcagcagtttctttccgctgatggcgcaacgtcgctggaagccggatcgcattattggcgtggtgccaacgctgttttgcgcgccgggaatgcgcctgctggcgaaactctctggtgcgcgtaccgtgctgcatattcaggattacgaagtggacgccatgctggggctgggccttgccggaaaaggcaaaggcggcaaagtggcacagctggcaacggcgttcgaacgtagcggactgcataacgtcgataacgtctccacgatttcgcgttcgatgatgaataaagccatcgaaaaaggcgtggcggcggaaaacgtcatcttcttccccaactggtcggaaattgcccgttttcagcatgttgcagatgccgatgttgatgcccttcgtaaccagcttgacctgccggataacaaaaaaatcattctttactccggcaatattggtgaaaagcaggggctggaaaacgttattgaagctgccgatcgtctgcgcgatgaaccgctgatttttgccattgtcgggcagggcggcggcaaagcgcggctggaaaaaatggcgcagcagcgtggactgcgcaacatgcaatttttcccgctgcaatcgtatgacgctttacccgcactgctgaagatgggcgattgccatctggtggtgcaaaaacgcggcgcggcagatgccgtattgccgtcgaaactgaccaatattctggcagtaggcggtaacgcggtgattactgctgaagcctacacagaactggggcagctttgcgaaacctttccgggcattgcggtttgcgttgaaccggaatcggtcgaggcgctggtggcggggatccgtcaggcgctcctgctgcccaaacacaacacggtggcacgtgaatatgccgaacgcacgctcgataaagagaacgtgttacgtcaatttataaatgatattcggggataattatggcgcagtcgaaactctatccagttgtgatggcaggtggctccggtagccgcttatggccgctttcccgcgtactttatcccaagcagtttttatgcctgaaaggcgatctcaccatgctgcaaaccaccatctgccgcctgaacggcgtggagtgcgaaagcccggtggtgatttgcaatgagcagcaccgctttattgtcgcggaacagctgcgtcaactgaacaaacttaccgagaacattattctcgaaccggcagggcgaaacacggcacctgccattgcgctggcggcgctggcggcaaaacgtcatagcccggagagcgacccgttaatgctggtattggcggcggatcatgtgattgccgatgaagacgcgttccgtgccgccgtgcgtaatgccatgccatatgccgaagcgggcaagctggtgaccttcggcattgtgccggatctaccagaaaccggttatggctatattcgtcgcggtgaagtgtctgcgggtgagcaggatatggtggcctttgaagtggcgcagtttgtcgaaaaaccgaatctggaaaccgctcaggcctatgtggcaagcggcgaatattactggaacagcggtatgttcctgttccgcgccggacgctatctcgaagaactgaaaaaatatcgcccggatatcctcgatgcctgtgaaaaagcgatgagcgccgtcgatccggatctcaattttattcgcgtggatgaagaagcgtttctcgcctgcccggaagagtcggtggattacgcggtcatggaacgtacggcagatgctgttgtggtgccgatggatgcgggctggagcgatgttggctcctggtcttcattatgggagatcagcgcccacaccgccgagggcaacgtttgccacggcgatgtgattaatcacaaaactgaaaacagctatgtgtatgctgaatctggcctggtcaccaccgtcggggtgaaagatctggtagtggtgcagaccaaagatgcggtgctgattgccgaccgtaacgcggtacaggatgtgaaaaaagtggtcgagcagatcaaagccgatggtcgccatgagcatcgggtgcatcgcgaagtgtatcgtccgtggggcaaatatgactctatcgacgcgggcgaccgctaccaggtgaaacgcatcaccgtgaaaccgggcgagggcttgtcggtacagatgcaccatcaccgcgcggaacactgggtggttgtcgcgggaacggcaaaagtcaccattgatggtgatatcaaactgcttggtgaaaacgagtccatttatattccgctgggggcgacgcattgcctggaaaacccggggaaaattccgctcgatttaattgaagtgcgctccggctcttatctcgaagaggatgatgtggtgcgtttcgcggatcgctacggacgggtgtaaacgtcgcatcaggcaatgaatgcgaaaccgcggtgtaaataacgacaaaaataaaattggccgcttcggtcagggccaactattgcctgaaaaagggtaacgatatgaaaaaattaacctgctttaaagcctatgatattcgcgggaaattaggcgaagaactgaatgaagatatcgcctggcgcattggtcgcgcctatggcgaatttctcaaaccgaaaaccattgtgttaggcggtgatgtccgcctcaccagcgaaaccttaaaactggcgctggcgaaaggtttacaggatgcgggcgttgacgtgctggatattggtatgtccggcaccgaagagatctatttcgccacgttccatctcggcgtggatggcggcattgaagttaccgccagccataatccgatggattataacggcatgaagctggttcgcgagggggctcgcccgatcagcggagataccggactgcgcgacgtccagcgtctggctgaagccaacgactttcctcccgtcgatgaaaccaaacgcggtcgctatcagcaaatcaacctgcgtgacgcttacgttgatcacctgttcggttatatcaatgtcaaaaacctcacgccgctcaagctggtgatcaactccgggaacggcgcagcgggtccggtggtggacgccattgaagcccgctttaaagccctcggcgcgcccgtggaattaatcaaagtgcacaacacgccggacggcaatttccccaacggtattcctaacccactactgccggaatgccgcgacgacacccgcaatgcggtcatcaaacacggcgcggatatgggcattgcttttgatggcgattttgaccgctgtttcctgtttgacgaaaaagggcagtttattgagggctactacattgtcggcctgttggcagaagcattcctcgaaaaaaatcccggcgcgaagatcatccacgatccacgtctctcctggaacaccgttgatgtggtgactgccgcaggtggcacgccggtaatgtcgaaaaccggacacgcctttattaaagaacgtatgcgcaaggaagacgccatctatggtggcgaaatgagcgcccaccattacttccgtgatttcgcttactgcgacagcggcatgatcccgtggctgctggtcgccgaactggtgtgcctgaaagataaaacgctgggcgaactggtacgcgaccggatggcggcgtttccggcaagcggtgagatcaacagcaaactggcgcaacccgttgaggcgattaaccgcgtggaacagcattttagccgtgaggcgctggcggtggatcgcaccgatggcatcagcatgacctttgccgactggcgctttaacctgcgcacctccaataccgaaccggtggtgcgcctgaatgtggaatcgcgcggtgatgtgccgctgatggaagcgcgaacgcgaactctgctgacgttgctgaacgagtaa

SEQ ID NO: 31 - DNA, 909 bp, Helicobacter pylori 26695
atggcgttcaaagtggtccaaatctgcggtggtctgggtaatcaaatgttccaatatgccttcgctaaatcgctgcaaaaacacagtaataccccggtcctgctggatattacgagttttgattggtccgaccgtaaaatgcagctggaactgttcccgattgatctgccgtatgcgagcgccaaagaaatcgcaattgctaaaatgcagcatctgccgaaactggttcgtgatgcgctgaaatgcatgggctttgaccgcgtcagtcaagaaatcgtgttcgaatatgaaccgaaactgctgaaaccgtcccgtctgacctatttctttggttactttcaggacccgcgttacttcgacgccatctctccgctgattaaacaaacctttacgctgccgccgccgccggaaaacaacaaaaacaacaacaaaaaagaagaagaatatcagtgcaaactgagcctgatcctggcggccaaaaactctgtgtttgttcacattcgtcgcggcgattacgtgggcatcggttgtcagctgggtattgactatcagaaaaaagcgctggaatacatggccaaacgtgttccgaatatggaactgtttgtcttctgcgaagatctggaatttacccaaaacctggacctgggctatccgttcatggatatgaccacgcgcgacaaagaagaagaagcgtattgggatatgctgctgatgcagagctgtcaacatggtattatcgctaatagcacgtattcttggtgggcagcttacctgattgaaaacccggaaaaaattatcattggcccgaaacattggctgtttggtcacgaaaatatcctgtgtaaagaatgggtgaaaatcgaatcacacttcgaagttaaatcgcagaaatataacgcgctgggctaa

SEQ ID NO: 32 - DNA, 1278 bp, Helicobacter pylori 26695
atgttccaaccgctgctggacgcgttcatcgaatctgcctctattgaaaaaatggcctcgaaatcgccgccgccgccgctgaaaatcgcagtggctaattggtggggtgatgaagaaatcaaagaatttaaaaaatttgtgctgtatttcattctgtctcagcgttacgcaatcaccctgcatcaaaacccgaatgaatttagtgacctggtcttctccaacccgctgggtgcagcacgtaaaattctgagctatcagaataccaaacgcgtgttttacacgggtgaaaacgaatctccgaactttaacctgtttgattatgccatcggctttgatgaactggacttcaatgatcgttatctgcgcatgccgctgtattacaatgagctgcacattaaagctgaactggttaatgataccacggcgccgtataaactgaagggtaacagtctgtacgccctgaaaaaaccgtcccatcactttaaagaaaaccacccgaatctgtgcgcggtggttaacgacgaaagcgatctgctgaaacgtggctttgcatcattcgttgcttcgaacgcgaatgccccgatgcgcaacgcgttttatgatgccctgaacagcattgaaccggttaccggcggtggctcggtccgtaatacgctgggttgcaaagtgggcaacaaaagcgaatttctgtctcagtacaaattcaacctgtgtttcgaaaatagtcaaggttatggctacgttaccgaaaaaatcctggatgcgtatttctcccatacgattccgatctactggggtagcccgtctgtcgccaaagattttaacccgaaatctttcgtcaatgtgcacgacttcaacaacttcgacgaagcaatcgattacatcaaatacctgcatacccacccgaatgcttatctggatatgctgtacgaaaacccgctgaatacgctggacggcaaagcctatttttaccaggatctgagtttcaagaaaattctggatttctttaaaaccattctggaaaacgatacgatctaccataaattcagtaccagctttatgtgggaatacgacctgcacaaaccgctggttagcatcgatgacctgcgtgttaactatgatgacctgcgtgtcaattacgatcgcctgctgcagaacgcatcaccgctgctggaactgtcgcaaaataccacgtttaaaatctatcgcaaagcgtatcaaaaatcactgccgctgctgcgtgctgtccgtaaactggtcaaaaaactgggtctgtaa

SEQ ID NO: 33 - DNA, 1179 bp, Escherichia coli
atgatctggataatgacgatggctcgccgtatgaacggtgtttacgcggcatttatgctggtcgcttttatgatgggggtggccggggcgctacaggctcctacattgagcttatttctgagtcgtgaggttggcgcgcaacctttctggatcggcctcttttatacggtgaatgctattgctgggatcggcgtaagcctctggttggcaaaacgttctgacagtcagggcgatcggcgaaaactgattatattttgctgtttgatggctatcggcaatgcgctattgtttgcatttaatcgtcattatctgacgcttatcacctgtggtgtgcttctggcatctctggccaatacggcaatgccacagttatttgctctggcgcgggaatatgcggataactcggcgcgagaagtggtgatgtttagctcggtgatgcgtgcgcagctttctctggcatgggttatcggtccaccgttggcctttatgctggcgttgaattacggctttacggtgatgttttcgattgccgccgggatattcacactcagtctggtattgattgcatttatgcttccgtctgtggcgcgggtagaactgccgtcggaaaatgctttatcaatgcaaggtggctggcaggatagtaacgtacggatgttatttgtcgcctcgacgttaatgtggacctgcaacaccatgtacattattgatatgccgttgtggatcagtagcgagttaggattgccagacaaactggcgggtttcctgatggggacggcagctggactggaaataccagcaatgattctggctggctactatgtcaaacgttatggtaagcggcgaatgatggtcatagcagtggcggcaggagtactgttttacaccggattgattttctttaatagccgtatggcgttgatgacgctgcaactttttaacgctgtatttatcggcattgttgcgggtattgggatgctatggtttcaggatttaatgcctggaagagcgggggcagctaccaccttatttactaacagtatttctaccggggtaattctggctggcgttattcagggagcaattgcacaaagttgggggcactttgctgtctactgggtaattgcggttatttctgttgtcgcattatttttaaccgcaaaggttaaagacgtttga

SEQ ID NO: 34 - DNA, 1197 bp, Serratia marcescens
atgcagcgtctgagccgtctgagcctgcgtatcaacccgattttcgcggcgtttctgctgatcgcgttcctgagcggtattgcgggtgcgctgctgaccccgaccctgagcctgtttctgaccaccgaggtgaaggttcgtccgctgtgggtgggtctgttctacaccgcgaacgcggttgcgggcatcgtggttagctttctgctggcgaaacgtagcgacacccgtggtgaccgtcgtcgtctgattctgctgtgctgcctgatggcggtgggcaactgcctgctgttcgcgtttaaccgtgactacctgaccctgatcaccgcgggtgtgctgatgagcgcggttgcgaacaccgcgatgccgcagattttcgcgctggcgcgtgaatatgcggatagcgaggcgcgtgaagtggttatgtttagcagcgtgatgcgtgcgcaactgagcctggcgtgggttattggtccgccgctgagcttcgcgctggcgctgaactatggcttcaccgtgatgtttctgattgcggcggttaccttcgcggtgtgcgttctgctggttggttttatgctgccgagcgttccgcgtgcggcggagaacgaaggcctgcagggtggcgtgagcgcgccgattgcgccggcgagcgcgtggcgtaaccgtgacgttcgtctgctgtttattgcgagcatgctgatgtggacctgcaacaccctgtacatcattgacatgccgctgtatatcaccgcggatctgggtctgccggagggtctggcgggcgtgctgatgggcaccgcggcgggcctggaaatcccggcgatgctgctggcgggttactatgttaagcgtttcggcaaacgtaacatgatgctgctggcggtggttgcgggtgtgctgttttacctgggcctgaccgttctggagagcaaaccggcgctgattgcgctgcagctgctgaacgcggtgttcatcggtattgttgcgggtattggcatgctgtattttcaggacctgatgccgggtcgtccgggtgcggcgaccaccctgttcaccaacagcatcagcaccggcgtgattctggcgggtgttctgcaaggcgcgctggttgagaacctgggtcacggcagcgtttactggatggcggcgctgctggcgctggcggcgctgggtatgagcgcgaaagtgcgtgaagtttaa

SEQ ID NO: 35 - DNA, 1185 bp, Rosenbergiella nectarea
atgcagagcttcaccccgccggcgccgaagggtggcaacccggtgttcatgatgtttatgctggtgaccttctttgtgagcattgcgggtgcgctgcaggcgccgaccctgagcctgtacctgagccaagagctggcggcgaaaccgttcatggtgggcctgttctttaccattaacgcggttaccggtatcattatcagctttatcctggcgaagcgtagcgaccgtaaaggtgaccgtcgtcgtctgctgatgttctgctgcgcgatggcgatcgcgaacgcgctgatgttcgcgtttgttcgtcagtacgtggttctgattaccctgggcctgatcctgagcgcgctgaccagcgtggttatgccgcaactgttcgcgctggcgcgtgagtatgcggaccgtaccggtcgtgaagtggttatgtttagcagcgtgatgcgtacccaaatgagcctggcgtgggttattggcccgccgatcagcttcgcgctggcgctgaactacggttttattaccctgtatctggtggctgcggcgctgtttctgctgagcctgattctgatcaagaccaccctgccgagcgttccgcgtctgtatccggcggaagacctggcgaagagcgcggcgagcggttggaaacgtaccgatgtgcgtttcctgtttgcggcgagcgtgctgatgtgggtttgcaacctgatgtacattatcgatatgccgctgtatatcagcaaaagcctgggtatgccggagagcttcgcgggtgttctgatgggcaccgcggcgggtctggaaattccggtgatgctgctggcgggctacctggcgaagcgtgttggtaaacgtccgctggtgattgttgcggcggtgtgcggcctggcgttctatccggcgatgctggtttttcaccagcaaaccggtctgctgattatccagctgctgaacgcggtgttcattggcatcgtggcgggtctggttatgctgtggtttcaagacctgatgccgggtaaagcgggtgcggcgaccaccctgttcaccaacagcgttagcaccggcatgatctttgcgggcctgtgccagggtctgctgagcgatctgctgggtcaccaagcgatttacgtgctggcgaccgtgctgatggttatcgcgctgctgctgctgctgcgtgttaaagagcaggcgtaa

SEQ ID NO: 36 - DNA, 1179 bp, Pantoea vagans
atgaagagcctgctgacccgtaaacgtcgtattaacccggtgttcctggcgtttatggcggcgagcttcatgatcggtgttgcgggtgcgctgcaggcgccgaccctgagcctgtttctgacccgtgaggtgcaagcgcgtccgctgtgggtgggcctgttctttaccgttaacgcgatcgcgggtattgtggttagcatgctggttgcgaagcgtagcgacagccgtggcgatcgtcgtaccctgattctgttctgctgcgcgatggcgttttgcaacgcgctgctgttcgcgtttacccgtcactacctgaccctgattaccctgggtgtgctgctgagcgcgctggcgagcgttagcatgccgcagattttcgcgctggcgcgtgagtatgcggaccaaagcgcgcgtgaagcggtgatgtttagcagcgttatgcgtgcgcagctgagcctggcgtgggtgattggcccgccgctgagcttcgcgctggcgctgaacttcggttttgtgaccctgttcctggttgctgcggcgctgtttctggtgtgcatcctgctgattaagtttaccctgccgagcgttccgcgtgcggaaccgctgatgcgtagcggtggcatgccgctgagcggttggcgtgaccgtgatgtgcgtctgctgttcattgcgagcgttaccatgtggacctgcaacaccatgtacatcattgacatgccgctgtatatcagcgttaccctgggtctgccggagaaactggcgggtctgctgatgggcaccgcggcgggtctggaaattccggtgatgctgctggcgggtcactatgcgaagcgtgttggtaaacgtaacctgatgctgattgcggtggcggcgggcgttctgttctatgcgggtctggcgatgtttgcgagccagaccgcgctgatggcgctgcaactgttcaacgcggtgtttattggcatcattgcgggtatcggcatgctgtggttccaggatctgatgccgggtcgtccgggtgcggcgaccaccatgtttaccaacagcatcagcaccggtatgattctggcgggcgttatccaaggcaccctgagcgagcgtttcggccacattgcggtgtattggctggcgctgggtctggcggttgcggcgtttgcgatgagcgcgcgtgtgaaaaacgtttaa

SEQ ID NO: 37 - protein, 302 aa, Helicobacter pylori 26695
MAFKVVQICGGLGNQMFQYAFAKSLQKHSNTPVLLDITSFDWSDRKMQLELFPIDLPYASAKEIAIAKMQHLPKLVRDALKCMGFDRVSQEIVFEYEPKLLKPSRLTYFFGYFQDPRYFDAISPLIKQTFTLPPPPENNKNNNKKEEEYQCKLSLILAAKNSVFVHIRRGDYVGIGCQLGIDYQKKALEYMAKRVPNMELFVFCEDLEFTQNLDLGYPFMDMTTRDKEEEAYWDMLLMQSCQHGIIANSTYSWWAAYLIENPEKIIIGPKHWLFGHENILCKEWVKIESHFEVKSQKYNALG

SEQ ID NO: 38 - protein, 425 aa, Helicobacter pylori 26695
MFQPLLDAFIESASIEKMASKSPPPPLKIAVANWWGDEEIKEFKKSVLYFILSQRYAITLHQNPNEFSDLVFSNPLGAARKILSYQNTKRVFYTGENESPNFNLFDYAIGFDELDFNDRYLRMPLYYAHLHYKAELVNDTTAPYKLKDNSLYALKKPSHHFKENHPNLCAVVNDESDLLKRGFASFVASNANAPMRNAFYDALNSIEPVTGGGSVRNTLGYKVGNKSEFLSQYKFNLCFENSQGYGYVTEKILDAYFSHTIPIYWGSPSVAKDFNPKSFVNVHDFNNFDEAIDYIKYLHTHPNAYLDMLYENPLNTLDGKAYFYQDLSFKKILDFFKTILENDTIYHKFSTSFMWEYDLHKPLVSIDDLRVNYDDLRVNYDRLLQNASPLLELSQNTTFKIYRKAYQKSLPLLRAVRKLVKKLGL

SEQ ID NO: 39 - protein, 425 aa, Artificial
  FutA_mut4
MFQPLLDAFIESASIEKMASKSPPPPLKIAVANWWGDEEIKEFKKFVLYFILSQRYAITLHQNPNEFSDLVFSNPLGAARKILSYQNTKRVFYTGENESPNFNLFDYAIGFDELDFNDRYLRMPLYYNELHIKAELVNDTTAPYKLKGNSLYALKKPSHHFKENHPNLCAVVNDESDLLKRGFASFVASNANAPMRNAFYDALNSIEPVTGGGSVRNTLGCKVGNKSEFLSQYKFNLCFENSQGYGYVTEKILDAYFSHTIPIYWGSPSVAKDFNPKSFVNVHDFNNFDEAIDYIKYLHTHPNAYLDMLYENPLNTLDGKAYFYQDLSFKKILDFFKTILENDTIYHKFSTSFMWEYDLHKPLVSIDDLRVNYDDLRVNYDRLLQNASPLLELSQNTTFKIYRKAYQKSLPLLRAVRKLVKKLGL

SEQ ID NO: 40 - protein, 478 aa, Helicobacter pylori NCTC 11639
MFQPLLDAYVESASIEKMASKSPPPLKIAVANWWGDEEIKEFKNSVLYFILSQRYTITLHQNPNEFSDLVFGNPLGSARKILSYQNAKRVFYTGENESPNFNLFDYAIGFDELDFNDRYLRMPLYYDRLHHKAESVNDTTAPYKLKDNSLYALKKPSHCFKEKHPNLCAVVNDESDPLKRGFASFVASNPNAPIRNAFYDALNSIEPVTGGGSVRNTLGYNVKNKNEFLSQYKFNLCFENTQGYGYVTEKIIDAYFSHTIPIYWGSPSVAKDFNPKSFVNVHDFKNFDEAIDYIKYLHTHKNAYLDMLYENPLNTLDGKAYFYQNLSFKKILAFFKTILENDTIYHDNPFIFCRDLNEPLVTIDDLRVNYDDLRVNYDDLRINYDDLRVNYDDLRINYDDLRVNYDDLRVNYDDLRINYDDLRVNYDDLRVNYERLLSKATPLLELSQNTTSKIYRKAYQKSLPLLRAIRRWVKKLGL

SEQ ID NO: 41 - protein, 392 aa, Escherichia coli
MIWIMTMARRMNGVYAAFMLVAFMMGVAGALQAPTLSLFLSREVGAQPFWIGLFYTVNAIAGIGVSLWLAKRSDSQGDRRKLIIFCCLMAIGNALLFAFNRHYLTLITCGVLLASLANTAMPQLFALAREYADNSAREVVMFSSVMRAQLSLAWVIGPPLAFMLALNYGFTVMFSIAAGIFTLSLVLIAFMLPSVARVELPSENALSMQGGWQDSNVRMLFVASTLMWTCNTMYIIDMPLWISSELGLPDKLAGFLMGTAAGLEIPAMILAGYYVKRYGKRRMMVIAVAAGVLFYTGLIFFNSRMALMTLQLFNAVFIGIVAGIGMLWFQDLMPGRAGAATTLFTNSISTGVILAGVIQGAIAQSWGHFAVYWVIAVISVVALFLTAKVKDV

SEQ ID NO: 42 - protein, 393 aa, Yersinia frederiksenii
MKSALTFSRRINPVFLAFFVVAFLSGIAGALQAPTLSLFLSTEVKVRPLWVGLFYTVNAIAGITVSFVLAKRSDLRGDRRKLILVCYLMAVGNCLLFAFNRDYLTLITAGVLLAAVANTAMPQIFALAREYADNSAREVVMFSSIMRAQLSLAWVIGPPLSFMLALNYGFTLMFCIAAGIFVLSALVVWFILPSVQRAEPVMDAPTVAQGSLFADKDVLLLFIASMLMWTCNTMYIIDMPLYITASLGLPERLAGLLMGTAAGLEIPIMLLAGYSVRRFGKRKIMLFAVLAGVLFYTGLVLFKFKSALMLLQIFNAIFIGIVAGIGMLYFQDLMPGRAGAATTLFTNSISTGVILAGVLQGVLTETWGHNSVYVMAMILAILSLIICARVREA

SEQ ID NO: 43 - protein, 387 aa, Rouxiella badensis
MSSRRLSIIFATFLLVSFLTGIAGALQAPTLSLFLTNEVKVRPLWVGLFYTVNALGGIVISFLLANYSDKKGDRRKLLFFCTLMAIGNSLIFAYSRDYLVLISVGVLLAAIGNASMPQLFALAREYADRSAHEVVMFSSMMRATLSLAWVLGPPISFTLALNYGFTLMYLCAAGVFIFSALMVWFFLPSVGRIEQPVDKVVVHVSAWKNRDVRLLFFASLLMWTCNIMYIIDMPLYITSDLGLPEGLAGLLMGAAAGLEIPVMLIAGYLVKRTGKRRLMLCAAVFGILFYLGLVLFQFKAALMILQLFNAIFIGIIAGIGMLYFQDLMPGRAGSATTLFTNSISTGAILAGVIQGTIVQNFGHYQVYWMALALAVGALVLMTRVKNV

SEQ ID NO: 44 - protein, 394 aa, Yersinia bercovieri; identical to SEQ ID NO: 2

SEQ ID NO: 45 - DNA, 1182 bp, Yersinia frederiksenii
atgaagagcgcgctgaccttcagccgtcgtattaacccggtttttctggcgttctttgtggttgcgttcctgagcggtattgcgggtgcgctgcaggcgccgaccctgagcctgttcctgagcaccgaggtgaaagttcgtccgctgtgggtgggcctgttctacaccgttaacgcgattgcgggtatcaccgtgagctttgttctggcgaagcgtagcgacctgcgtggcgatcgtcgtaaactgatcctggtgtgctacctgatggcggttggtaactgcctgctgttcgcgtttaaccgtgactatctgaccctgattaccgcgggcgtgctgctggcggcggttgcgaacaccgcgatgccgcagattttcgcgctggcgcgtgagtacgcggataacagcgcgcgtgaagtggttatgtttagcagcattatgcgtgcgcaactgagcctggcgtgggttatcggtccgccgctgagcttcatgctggcgctgaactatggcttcaccctgatgttttgcattgcggcgggtatcttcgtgctgagcgcgctggttgtgtggtttattctgccgagcgtgcagcgtgcggaaccggttatggatgcgccgaccgtggcgcaaggcagcctgttcgcggacaaggatgttctgctgctgtttattgcgagcatgctgatgtggacctgcaacaccatgtacatcattgatatgccgctgtatatcaccgcgagcctgggtctgccggagcgtctggcgggtctgctgatgggcaccgcggcgggtctggaaatcccgattatgctgctggcgggctacagcgtgcgtcgttttggcaagcgtaaaatcatgctgttcgcggtgctggcgggcgttctgttttataccggtctggttctgttcaagtttaaaagcgcgctgatgctgctgcagattttcaacgcgatctttattggtatcgtggcgggtatcggcatgctgtacttccaagacctgatgccgggtcgtgcgggtgcggcgaccaccctgtttaccaacagcattagcaccggcgttatcctggcgggcgtgctgcaaggtgttctgaccgaaacctggggtcacaacagcgtgtatgttatggcgatgattctggcgatcctgagcctgatcatttgcgcgcgtgtgcgtgaagcgtaa

SEQ ID NO: 46 - DNA, 1164 bp, Rouxiella badensis
atgagcagccgtcgtctgagcatcattttcgcgacctttctgctggttagcttcctgaccggtattgcgggtgcgctgcaggcgccgaccctgagcctgtttctgaccaacgaggttaaagtgcgtccgctgtgggttggtctgttctacaccgtgaacgcgctgggtggcatcgttattagctttctgctggcgaactatagcgacaagaaaggtgatcgtcgtaaactgctgttcttttgcaccctgatggcgatcggcaacagcctgattttcgcgtacagccgtgactatctggtgctgatcagcgttggtgtgctgctggcggcgattggcaacgcgagcatgccgcagctgtttgcgctggcgcgtgagtacgcggatcgtagcgcgcacgaagtggttatgtttagcagcatgatgcgtgcgaccctgagcctggcgtgggttctgggtccgccgatcagcttcaccctggcgctgaactacggttttaccctgatgtatctgtgcgcggcgggcgtgttcatctttagcgcgctgatggtttggttctttctgccgagcgtgggccgtattgaacaaccggttgacaaggtggttgtgcacgtgagcgcgtggaaaaaccgtgatgttcgtctgctgttctttgcgagcctgctgatgtggacctgcaacatcatgtacatcattgacatgccgctgtatattaccagcgatctgggtctgccggagggtctggcgggcctgctgatgggtgctgcggcgggcctggaaatcccggttatgctgattgcgggttacctggtgaagcgtaccggcaaacgtcgtctgatgctgtgcgcggcggttttcggtatcctgttttatctgggcctggtgctgttccagtttaaggcggcgctgatgatcctgcaactgttcaacgcgatctttattggtatcattgcgggtattggcatgctgtatttccaggacctgatgccgggtcgtgcgggcagcgcgaccaccctgtttaccaacagcatcagcaccggcgcgattctggcgggtgttatccagggcaccattgtgcaaaacttcggtcactaccaagtgtattggatggcgctggcgctggcggttggtgcgctggtgctgatgacccgtgttaaaaacgtgtaa

SEQ ID NO: 47 - DNA, 1185 bp, Yersinia bercovieri; identical to SEQ ID NO: 35

SEQ ID NO: 48 - DNA, 31 bp, Artificial Sequence
  KABY733 fred.for
aaacagcuatgaagagcgcgctgaccttcag

SEQ ID NO: 49 - DNA, 29 bp, Artificial
  KABY734 fred.rev
agggttaauttacgcttcacgcacacgcg

SEQ ID NO: 50 - DNA, 29 bp, Artificial
  KABY729 bad.for
aaacagcuatgagcagccgtcgtctgagc

SEQ ID NO: 51 - DNA, 36 bp, Artificial
  KABY730 bad.rev
agggttaauttacacgtttttaacacgggtcatcag

SEQ ID NO: 52 - DNA, 32 bp, Artificial
  KABY721 yberc.for
aaacagcuatgaagagcgcgctgacctttagc

SEQ ID NO: 53 - DNA, 29 bp, artificial
  KABY722 yberc.rev
agggttaauttacgcctcacgcacacgcg

SEQ ID NO: 54 - protein, 608 aa, Acanthamoeba polyphaga moumouvirus
MDKFKIVCINLERRKDRKDLITNKLINQNINNFEFFEAIDGSKIDPDDERLNLFKHSVSGLLRRGVTGCALSHYTIWKKLINDPDYNTYLVIEDDINFGNDFKSALEKILDKSPGHGIVLLGMTLELEKRAETKHLYQYDTSYSVHNLSRDLYCGGAFGYIITKSAAEYLVGYISCNGIRIVIDYLMFRSGVPMYESHPHLVFTDAVQHSIHYVDSDIQHDYERIKYNKLLNNYQYDDYVFLSNRDSVYGDIREVCADIPTLKRAADMTPECVAFNTYGWLKNTLTDFDKFIVLHDKYYTHDGIYIKKSYFSLENKLKNLRLLERPIRIFLNNNTINYSEHLVNIILKNIPNYDIVKDNNDADIIIDNINDGKLFYDITKLNIIISGEPFNRKQKYDIAIDTKKNSNAEYTIYHPFLFSSLHEHKKSINYLDYVIPKTKFCAYMYHMSYPHRINYFNIISSYKHVDALGKCCNNVEIKNTRYVLNNKETYNDIAVEYFTQYKFVLAIENNMIPGYNTEKLINPMIANSIPIYWGDSEIFKYINKRRLVYIPDFATNEDLINHIKYIDEHDDVYENIIKESIFTDPNFTLDVIEQHLSKEIDNLFGLKN